(19) United States
(12) Patent Application Publication
(10) Pub. No.: US 2023/0122977 A1
(43) Pub. Date: Apr. 20, 2023

(54) Regenerative Tissue-Mimetic Multilayer Fused Microgel-Cell Construct

(71) Applicant: University of South Carolina, Columbia, SC (US)

(72) Inventor: Esmaiel Jabbari, Bethesda, MD (US)

(73) Assignee: University of South Carolina, Columbia, SC (US)

(21) Appl. No.: 17/691,262

(22) Filed: Mar. 10, 2022

Related U.S. Application Data
(60) Provisional application No. 63/187,996, filed on May 13, 2021.

(51) Int. Cl. A61L 27/36; C12N 5/074; A61K 9/51; A61L 27/38; A61L 27/54; A61L 27/56; A61L 27/22

(52) U.S. Cl. CPC A61L 27/3612; A61K 9/51; A61L 27/38; A61L 27/54; A61L 27/56; A61L 27/227; C12N 5/0607; A61L 2101/46; A61L 2400/12; A61L 2400/18; A61L 2430/10; A61L 2430/20; A61L 2430/26; A61L 2430/32; A61L 2430/34; A61L 2430/40

(57) ABSTRACT

Described herein are regenerative approaches with tunable cell-cell and cell-matrix interactions to enhance the ability to regenerate multiple zones within a construct with each zone possessing a unique, optimum, level of cell-cell and cell-matrix interaction.

### STATEMENT REGARDING FEDERALLY SPONSORED RESEARCH

This disclosure was made with government support under R56 ARO63745 awarded by the National Institute of Health, 1500242 by the National Science Foundation, and 1403545 by the National Science Foundation. The government may have certain rights in the invention.

### TECHNICAL FIELD

The subject matter disclosed herein is generally directed to regenerative approaches with tunable cell-cell and cell-matrix interactions to enhance the ability to regenerate multiple zones within a construct with each zone possessing a unique, optimum, level of cell-cell and cell-matrix interaction.

### BACKGROUND

Cartilage degeneration is particularly relevant in older populations, with 65% of those over 60 years old experiencing joint pain, causing a long-term disability and lower quality of life. In addition, immobile lifestyles caused by cartilage loss may cause up to a 24% increase in the likelihood of developing chronic heart disease, which is a leading cause of death. The complex physical and biochemical properties of articular cartilage have resulted in limited experimental and clinical success in replicating its structure and function, with few treatment options for those with cartilage loss.

Autologous Chondrocyte Implantation (ACI), a current treatment for cartilage injury, involves expanding chondrocytes in vitro, transplanting the expanded cells into a defect site, and holding them in place via a sutured periosteal flap. ACI studies show that the repaired tissue has similar biochemical properties to the native tissue and has better long-term outcomes than microfractures and other traditional methods. However, the procedure is dual staged, and there is often poor cell retention and dedifferentiation following implantation. Periosteal hypertrophy, ablation, leakage of chondrocytes, and nonhomogeneous cell distribution often occur after implantation, resulting in additional surgical requirements. These limitations reduce the potential of ACI as a treatment method. Additionally, during chondrocyte harvesting, inherent donor site morbidity may cause further cartilage loss. Thus, alternative methods have been explored for the repair of cartilage defect.

Currently, human cells are commercially expanded on cell culture bioreactors with synthetic microcarriers that do not mimic natural tissue. As these microcarriers are not natural to the expanded cells, the cells are detached and separated from the microcarriers, reseeded in a porous scaffold, and cultured in a bioreactor prior to implantation. The detachment of the cells from microcarriers and reseeding the cells in a scaffold negatively affects cell viability and functionality. Accordingly, there is a need to simplify the aforementioned process of cell expansion, separation, scaffold reseeding, and cultivation in bioreactors in order to increase cell viability and functionality, reduce contamination and cost, and enhance quality of the tissue regeneration implant.

Citation or identification of any document in this application is not an admission that such a document is available as prior art to the present disclosure.

### SUMMARY

The above objectives are accomplished according to the present disclosure by providing a method for forming a novel monolayer implant construct. The method may include forming at least one nanogel in at least one microcapsule via chain extending at least one first polyethylene oxide macromer with at least one lactide-glycolide, terminating at least one chain end with an acrylate functional group, crosslinking the at least one first polyethylene oxide macromer with at least one lactide-glycolide terminated on at least one chain end with an acrylate functional group with a second polyethylene oxide macromer with at least one lactide-glycolide terminated on at least one chain end with an acrylate functional group to form at least one nanogel, and conjugating at least one morphogen to the at least one nanogel to form at least one morphogen-encapsulated nanogel, forming at least one cartilage microparticle from articular cartilage, transferring the at least one cartilage microparticle to a cell culture bioreactor containing at least one cell culture medium wherein at least one cell adheres to the at least one cartilage microparticle, forming a suspension comprising the at least one cartilage microparticle with at least one cell adhered, the at least one morphogen-encapsulated nanogel, and at least one crosslinking agent in a tissue culture medium, and employing a cross-linking initiator to form a cross-linked monolayer implant. Further release of the at least one morphogen may be controlled via changing a composition of the nanogel to change a release duration of the at least one morphogen. Still, the articular cartilage may be harvested from frozen human cadaver or animal tissue. Yet further, the at least one cartilage microparticle is decellularized. Again, the at least one cartilage microparticle may range in size from 50 to 500 µm. Yet still, the at least one cell culture medium comprises at least one mesenchymal stem cell. Moreover, the suspension may be injected at a tissue injury site prior to employing the cross linking initiator.

In a further embodiment, a method for forming an implantable microgel-cell construct for a zonally structured tissue is provided. The method may include forming at least one nanogel in at least one microcapsule via chain extending at least one first polyethylene oxide macromer with at least one lactide-glycolide, terminating at least one chain end with an acrylate functional group, crosslinking the at least one first polyethylene oxide macromer with at least one lactide-glycolide terminated on at least one chain end with an acrylate functional group with a second polyethylene oxide macromer with at least one lactide-glycolide terminated on at least one chain end with an acrylate functional group to form at least one nanogel and conjugating at least one morphogen to the nanogel to form at least one morphogen-encapsulated nanogel, forming at least one microparticle, transferring the at least one microparticle to a cell culture bioreactor containing at least one cell culture medium wherein at least one cell adheres to the at least one microparticle, forming a first suspension comprising the at least one microparticle with at least one cell adhered and the at least one morphogen-encapsulated nanogel, allowing the first suspension to settle gravitationally on a surface and fuse via secretion of an extracellular matrix tor form a first monolayer, forming a second suspension comprising the at least one microparticle with at least one cell adhered and the at least one morphogen-encapsulated nanogel, and allowing the second suspension to settle gravitationally on a surface and fuse via secretion of an extracellular matrix or form a second monolayer atop the first monolayer to form a multilayer construct. Further, the first monolayer may range from 100 to 500 µm in thickness. Yet again, the first monolayer and the second monolayer may be formed with different extracelluar matrix compositions and/or different morphogen nanogels. Furthermore, the multilayer construct may comprise three or more monolayers. Still further, the at least three or more monolayers may be formed with different extracelluar matrix compositions and/or different morphogen nanogels. Yet still, the suspension may be transferred to a mold with a predefined shape. Further again, release of the at least one morphogen may be controlled via changing a composition of the nanogel to change a release duration of the at least one morphogen. Again further, the at least one microparticle may be decellularized. Further still, the at least one microparticle may range in size from 50 to 500 µm. Moreover, the at least one cell culture medium may comprise at least one mesenchymal stem cell.

In a still further embodiment, a novel multilayer cellular construct is provided. The multilayer cellular construct may include at least one decellularized cell-seeded fused microcarrier, wherein each layer of the at least one multilayer cellular construct mimics biochemical and cellular properties of at least one zone of a zonally structured tissue. Further, the decellularized cell-seeded fused microcarrier may include at least one cartilage microparticle. Still yet, the at least one cartilage microparticle may comprise either human or animal articular cartilage. Still further, the at least one cartilage microcarrier may ranges in size from 50 to 500 µm. Moreover, the at least one multilayer cellular construct maybe used to regenerate heart, skin, articular cartilage, blood vessel, nerve conduit, ligament and/or tendon tissue.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows a schematic representation for production of injectable or implantable CMP-MSCs.

FIG. 2 shows the size distribution of adult (a-c) and fetal (d-f) CMPs.

FIG. 3 shows growth of MSCs on the fetal (a) and adult (b) CMPs with incubation time in basal medium in a tissue culture bioreactor for CMP particle sizes of 90 µm (red), 190 µm (green) and 250 µm (blue).

FIG. 4 shows the live (green) and dead (red) fluorescent images of a randomly selected microparticle from fetal and adult CMP-MSCs with incubation time (1, 7, 14, 21 days) in basal medium for CMP particle sizes of 90, 190 and 250 µm.

FIG. 5 shows mRNA expression of select genetic markers.

FIG. 6 shows gelation time of alginate gels as a function of CaCl₂ concentration.

FIG. 7 shows mRNA expression of chondrogenic markers.

FIG. 8 shows images of monolayer fCMP-MSCs (blue), monolayer aCMP-MSCs (red), and bilayer faCMP-MSCs (purple) cell sheets after 2, 4 , and 6 days incubation.

FIG. 9 shows compressive modulus of aCMP-MSCs/Alg (left column) and fCMP-MSCs/Alg (right column).

FIG. 10 shows at Exhibit A Calcein AM (green) and EthD (red) fluorescent images of live and dead MSCs, respectively; Exhibit B shows Phalloidin and DAPI stained images showing cytoskeletal and nuclear compartments of MSCs.

FIG. 11 shows Alcian blue (a) and H&E (b) stained histological sections of CMP-MSCs/alg hydrogel (second column), CMP-MSCs/ml (third column) and CMP-MSCs/bl cell sheets.

FIG. 12 shows Table 1.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/-10% or less, +/-5% or less, +/-1% or less, and +/-0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt%,” and “wt. %,” which can be used interchangeably, indicate the percent by weight of a given component based on the total weight of a composition of which it is a component, unless otherwise specified. That is, unless otherwise specified, all wt% values are based on the total weight of the composition. It should be understood that the sum of wt% values for all components in a disclosed composition or formulation are equal to 100. Alternatively, if the wt% value is based on the total weight of a subset of components in a composition, it should be understood that the sum of wt% values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

In tissue engineering, progenitor cells are harvested from the patient, and expanded in three-dimensional microcarrier-based cell culture bioreactors. Next, the expanded cells are separated from the substrate, purified, and seeded in scaffolds, cultured in perfusion bioreactors to guide differentiation and maturation to the desired lineage and phenotype. Currently, human cells are commercially expanded on tissue culture bioreactors with synthetic microcarriers like polystyrene, dextran, or gelatin microbeads. These microcarrier-based substrates are synthetic and general in nature, meaning that the substrate does not mimic that of the natural tissue. As these microcarriers are not natural to the expanded cells, the cells are detached and separated from the microcarriers, reseeded in a porous scaffold, and cultured in a bioreactor prior to implantation. The detachment of the cells from microcarriers and reseeding the cells in a scaffold negatively affects cell viability and functionality.

All tissue engineered articular cartilage therapies fall into one of the following categories: (a) injectable cellular hydrogels, (b) implantable cell pellet, and (c) implantable cellular scaffolds. In injectable hydrogels, the progenitor cells are encapsulated within a natural/synthetic hydrogel matrix with high cell-matrix interaction but little to no cell-cell interaction. As there is extensive cell-cell contact in the superficial layer and deep layer, the injectable hydrogel fails to regenerate the superficial and deep zones of articular cartilage. In the cell pellet, there is extensive (almost 100%) contact between the cells in the pellet but there is complete lack of cell-matrix contact, which is beneficial to regeneration of the superficial zone. As there is extensive cell-matrix interaction and high compressive stiffness in the middle zone, the cell pellet fails to regenerate the compressively-loaded middle zone of articular cartilage. The implantable cellular scaffolds suffer from in homogenous cell seeding, insufficient cell to cell interaction, and incoordination between scaffold degradation and matrix formation. Therefore, regenerative approaches with tunable cell-cell and cell-matrix interactions could enhance our ability to regenerate multiple zones within a construct with each zone possessing a unique, optimum levels of cell-cell and cell-matrix interaction.

Articular cartilage serves as a good example of a multilayer, zonally structured tissue. The organization of articular cartilage is initiated from the early stages of embryonic development by condensation and lineage commitment of mesenchymal stem cells (MSCs) in the lateral plate mesoderm by up-regulation of transforming growth factor-ß1 (TGFß1), the master regulator of chondrogenesis. Following condensation, MSCs begin differentiating into zone-specific chondrocytes via spatiotemporal expression of multiple morphogens. First, the condensed MSCs differentiate into pre-chondrocytes by up-regulation of bone morphogenetic protein-7 (BMP7) via the activation of Wnt/ß-catenin pathway via Smad 2/3/4 and transcription factors TCF/Lef1. The early structure of fetal articular cartilage is remarkably similar to that of the superficial zone of adult tissue. Next, the pre-chondrocytes in the developing fetal cartilage differentiate and adopt a pre-hypertrophic morphology driven by localized expression of insulin growth factor-1 (IGF1) to generate the superficial and middle zones. Next, the pre-hypertrophic chondrocytes differentiate to hypertrophic chondrocytes by localized expression of Indian hedgehog (IHH) through a pathway independent of parathyroid hormone-related protein (PTHrP). The spatiotemporal gradients in BMP7, IGF1, and IHH further divides the developing fetal cartilage into three distinct zones, namely the superficial, middle, and calcified zones. The maturation of stratified structure of articular cartilage is not limited to prenatal period as it continues during infancy, childhood, adolescence, and adulthood. Therefore, there is a need to develop multilayer cellular constructs with each layer having a zone-specific cellularity, extracellular matrix composition, and growth factor for regeneration of microstructured human tissues.

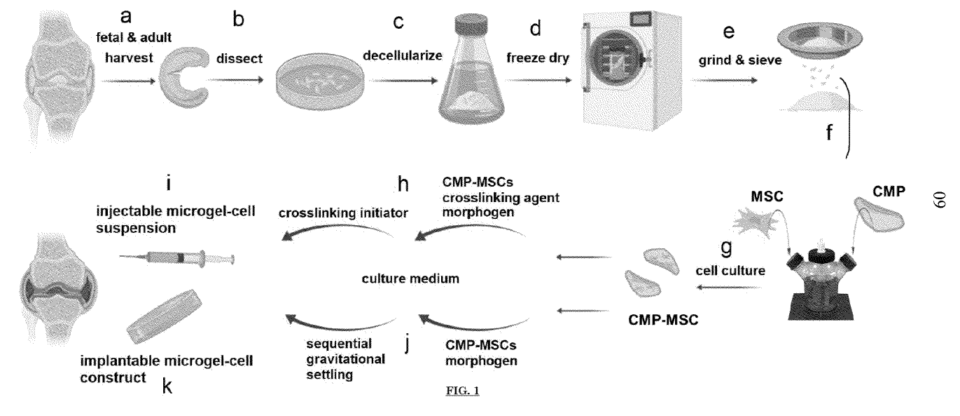

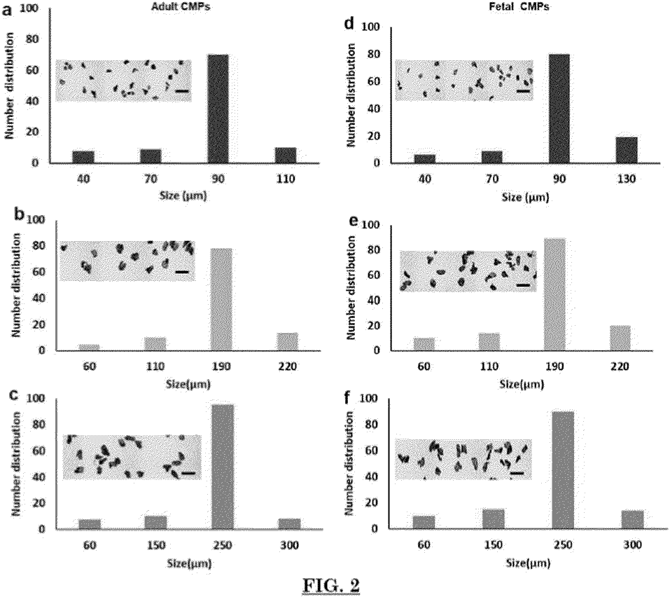

In this disclosure, a novel composition and process is developed to form multilayer cellular constructs, with each layer mimicking the biochemical and cellular properties of individual zones of articular cartilage, by sequential gravitational settling of cell-embedded, articular cartilage-based microcarriers. The process for production of a multilayer cellular construct using decellularized, cell-seeded, fused microcarriers, based on fetal or adult bovine articular cartilage tissue, is shown in FIGS. 1 and 2. The steps for production of a multilayer microgel-cell construct are as follows:
Step 1) First, nanogels in microcapsules are synthesized using the following procedure for morphogen stabilization and sustained release, as schematically demonstrated in FIG. 1. Polyethylene oxide macromers chain-extended with short lactide-glycolide and one chain-end terminated with an acrylate functional group (FIG. 1 at a) are self-assembled in aqueous solution (FIG. 1 at b) and cross-linked to form nanogels (PEG-bL-bG-AcNGs) (FIG. 1 at c). Next, the morphogen (growth factor, differentiation factor, maturation factor) is conjugated to the NGs (FIG. 1 at d). The duration of release of each morphogen can be controlled by the nanogel composition.
Step 2) Articular cartilage tissue is harvested from frozen human cadaver or animal tissue. The animal may be bovine or pig. The animal may be at different life stages including fetal, newborn, 6 months old, yearling, two years old, or an adult. The human cadaver may be at different stages of life including fetal, newborn, infant, early, middle, and late childhood, adolescent, or adult (FIG. 2 at a).
Step 3) The harvested articular cartilage is dissected and minced into pieces in the range of a few millimeter using techniques well known in the art (FIG. 2 at b).
Step 4) The minced articular cartilage is decellularized and genomic content removed using techniques and methods well known in the art. One such method is treatment with 10 mM Tris/1% triton with sonication followed by incubation in 1 U/mL deoxyribonuclease and 1 U/mL ribonuclease in phosphate buffer saline (FIG. 2 at c).
Step 5) The minced decellularized articular cartilage is freeze-dried or lyophilized using methods and techniques well known in the art to form a course powder (FIG. 2 at d).
Step 6) The freeze dried mined decellularized articular cartilage is grinded and sieved to produce a fine micronized powder with particle size in the range of 50 to 500 µm, using well known methods and techniques in the art. Preferably, the particle size should be in the 100 to 250 µm range. The decellularized articular cartilage microparticles are hereafter referred to as cartilage microparticles or CMPs (FIG. 2 at e).
Step 7) The CMPs are transferred to a cell culture bioreactor, like a stirred tank cell culture bioreactor, filled with the desired cell culture medium. The CMPs are allowed to hydrate and swell in the cell culture bioreactor. The cell culture bioreactor should be equipped with oxygen gas inlet and outlet for cell consumption, outlet for removal of carbon dioxide gas generated by cells, stirrer to maintain cells in suspension, and heating/cooling to maintain temperature of 37° C. Next, the desired cells are transferred to the cell culture bioreactor. The desired cells may be mesenchymal stem cells from the bone marrow or from the fat tissue of a patient harvested using well known methods in the art. The initial number of cells should be chosen such that there is at least one or more cells per CMP. The cells are allowed to adhere to surface of the CMP pores. After attachment, the cells are allowed to grow and expand number to many folds higher than the initial number of seeded cells. The MSCs attached and grown on CMPs are hereafter referred to as CMP-MSCs (FIG. 2 at f-g).
Step 8) Injectable suspension: In this approach, the CMP-MSCs and the morphogen-encapsulated NGs are suspended in tissue culture medium along with a crosslinking agent. The viscous suspension is injected into the site of tissue injury (defect), and the suspension is cross-linked using a crosslinking initiator, like visible or ultraviolet light or a chemical agent, to produce an in-situ cross-linked implant. This approach results in a monolayer implant (FIG. 2 at h-i).
Step 9) Implantable microgel-cell construct: In this approach, a suspension of CMP-MSCs and the morphogen NGs for a specified zone of the regenerating tissue in tissue culture medium is transferred to a sterile Teflon mold, the mold is placed in a sterile petri dish, and the assembly is incubated for 48 h. During incubation, the CMP-MSCs and morphogen NGs settle gravitationally on bottom surface of the mold and fuse by secretion of extracellular matrix (ECM) to form the first monolayer microgel-cell construct, hereafter referred to as CMPMSCs/ml-1. The thickness of the monolayer can range from 100-500 µm depending on CMP size. Next, the above process is repeated to form a bilayer, trilayer, or a multilayer microgel-cell construct with each layer having a unique cellularity, ECM composition, and morphogen NGs. It should be noted that in this process the morphogen NGs is trapped in the corresponding layer because the morphogen is attached to NGs and is unable to diffuse to other layers. For example, the CMP type, cellularity, and morphogen type in the first layer could be CMP from adult bovine articular cartilage, 15 M, and Indian Hedgehog (IHH) to simulate the calcified zone of articular cartilage; yearling bovine articular cartilage, 20 M, and insulin growth factor-1 (IGF-1) to simulate the middle zone; adult bovine articular cartilage, 60 M, and bone morphogenetic growth factor-7 (BMP-7) to simulate the superficial zone of articular cartilage (FIG. 1 at j-k).

The process described above for articular cartilage regeneration serves as an example and this invention is not limited to articular cartilage. CMPs generated from any animal or cadaver tissue can be used to culture and expand cells for regeneration of the same tissue in a human patient. The composition and process used to generate the multilayer microgel-cell construct can be used for regeneration of any zonally structured tissue.

Tissue engineering aims to make cellular constructs for implantation in an injured tissue defect in a human patient to restore, maintain, repair, regenerate or improve the function of the injured tissue. The process of engineering a tissue include a) harvesting cells that are capable of being grown or stem cells from the patient, b) expanding the harvested cells in a cell culture bioreactor using microcarriers as a substrate, c) detaching and separating the cells from the microcarrier substrate, d) making a porous scaffold with a geometry similar to that of the tissue defect, e) seeding the expanded cells and growth factors homogeneously inside the porous scaffold, f) guiding maturation of the seeded cells to the lineage of the tissue that is being repaired using tissue culture bioreactors, g) and implanting the construct (scaffold+cells+growth factor) in the tissue defect regenerating the injured tissue.

Tissue engineered constructs fall into one of the following categories: (a) injectable cellular hydrogels, (b) implantable cell pellets, and (c) implantable cellular scaffolds. In injectable hydrogels, the progenitor cells are encapsulated within a natural/synthetic hydrogel matrix with high cell-matrix interaction but little to no cell-cell interaction. In the cell pellet, there is extensive (almost 100%) contact between the cells in the pellet but there is lack of cell-matrix contact. The implantable cellular scaffolds suffer from in homogenous cell seeding, insufficient cell-cell interaction, and lack of synchronized scaffold degradation and matrix formation. Therefore, regenerative approaches with tunable cell-cell and cell-matrix interactions could enhance our ability to regenerate multiple zones within a construct with each zone possessing a unique, optimum level of cell-cell and cell-matrix interaction.

This disclosure describes a novel composition and process to form multilayer microgel-cell construct as constructs for implantation in a tissue defect using decellularized, micronized tissue particles as microcarriers for cell expansion as well as a scaffold for delivery to the site of injury. The decellularized, micronized tissue particles eliminate the undesired step of cell separation from microcarriers and reseeding the cells in a scaffold. Further, the decellularized, micronized tissue particles enable tuning and optimizing the extent of cell-cell and cell-matrix interactions. In addition, the decellularized, micronized tissue particles enable the production of multilayer microgel-cell constructs to mimic the zonal structure of regenerating tissue. The decellularized, micronized tissue particles can be used for regeneration of all human tissues from heart to skin, articular cartilage, blood vessels, nerve conduit, to ligament and tendon, among others.
1) Natural tissue-specific microcarriers for culture and expansion of human cells: Currently, human cells are commercially expanded on tissue culture bioreactors with synthetic microcarriers like polystyrene, dextran, or gelatin microbeads. These microcarrier-based cell substrates are synthetic and general in nature, meaning that the substrate does not mimic that of the natural tissue. As these microcarriers are not natural to the expanded cells, a three step process is using the cells for tissue regeneration. First, the harvested cells are expanded on the microcarriers; second, the cells are detached from the microcarriers; and third, the detached cells are seeded in a scaffold and cultured in a tissue bioreactor prior to implantation. The step of detaching cells from microcarriers and seeding them in a scaffold is very harmful to the cells and substantially reduces cell viability and functionality. In this disclosure, a composition and process is developed for the production of tissue-specific microcarriers that eliminates the step of cell detachment from microcarriers and seeding in a scaffold. In the proposed disclosure, the stem cells are expanded on microcarriers formed from the tissue that is being regenerated. For example, if the tissue to be regenerated is articular cartilage, the microcarriers are allogeneic, decellularized, micronized segments of articular tissue. Therefore, the expanded cells do not need to be detached from the microcarrier and the microcarrier-attached cells can be frozen until needed for implantation in the patient.
2) Injectable microcarrier-attached cell suspension for tissue regeneration: The microcarrier-attached cells can be used directly for cell delivery in clinical applications. For example, the suspension of microcarrier-attached cells, a crosslinking agent, and morphogen-loaded nanoparticles in physiological solution can be injected into an articular cartilage defect and cross-linked with light irradiation, using techniques and methods well known in the art, to form a cellular construct for tissue regeneration.
3) Gravitationally-formed implantable microgel-cell construct for tissue regeneration: The suspension of microcarrier-attached cells and morphogen-loaded nanoparticles in basal culture medium are transferred to a mold with predefined shape. As the microcarrier beads have microscale dimension in the order of 100-300 µm, the microbeads settle to the bottom surface of the mold by the force of gravity and form a cell layer. The cell layer is cultured in tissue-specific culture medium, like chondrogenic medium for articular cartilage, to fuse the microcarrier-cell layer into a solid cellular construct. In the process of fusion, the morphogen-loaded nanoparticles become trapped in the solid microgel-cell construct. The above procedure can be repeated many times with different types of microcarriers, different cell types, and different morphogens to form multilayer microgel-cell constructs with each layer representing a specified zone of the tissue being regenerated.
4) The disclosed composition and process can be used in regeneration of living tissues with spatially varying cell and morphogen content. One specific application is regeneration of articular cartilage with its stratified structure for patients with post-traumatic osteoarthritis (PTOA). About 3.5 million individuals in US suffer from posttraumatic osteoarthritis (PTOA) of the hip, knee, or ankle with a total cost of $3 billion to the health care system.

Approximately 12% of all osteoarthritis cases are caused by PTOA. PTOA can occur in any age from any acute physical trauma such as sport, vehicle accident, fall, or military injury. PTOA results in some form of permanent disability in 28% of patients with traumatic skeletal injuries. The rapidly applied load in traumatic injuries focally disrupts the articular cartilage beginning with cell death, inadequate synthesis of proteoglycans, and disturbed proteoglycan to water ratio, which leads to decreased stiffness and accelerated damage to the remainder of the joint. PTOA is diagnosed on average 19 months following traumatic injury such that the focal articular disruption has advanced to a full-thickness defect.

Conventional clinical approaches to treat full-thickness chondral defects such as debridement, lavage, subchondral drilling, microfracture, and abrasion arthroplasty create mechanically-inferior fibrocartilage.

Osteochondral autograft transfer or mosaicplasty suffers from an additional surgical intervention and donor site morbidity. Autologous chondrocyte implantation (ACI) reduces fibrocartilage formation but it fails to restore zonal organization of the articular cartilage and in some cases leads to peripheral hypertrophy and calcification. For example, 33% of ACI procedures performed on patients suffering from PTOA of the knee resulted in permanent disability. Recently, injection or implantation of a cellular matrix with bulk properties mimicking those of the thickest zone of the native articular cartilage (middle and deep zones) to regenerate the injured tissue has been reported. However, regenerative approaches that do not take into consideration the zonal structure of articular cartilage lead to degeneration into inferior fibrocartilage after implantation, failure of the therapy and patient rehospitalization.

The articular cartilage is bounded in the thickness direction by the synovial fluid next to the superficial layer and by the bone tissue next to the calcified layer. As a result of proximity to the synovial fluid on one side and the bone tissue on the other side, articular cartilage has a stratified structure in which the chemical composition, cellularity, and mechanical stiffness varies continuously from the superficial layer to the calcified layer. The stratified structure of articular cartilage can be divided roughly into zones including the superficial zone (in direct contact with the synovial fluid), transition zone, middle zone, deep zone, and final the calcified zone. Each zone is defined by its unique chemical composition, cellularity, morphogens, and mechanical properties. The superficial zone is characterized by pre-chondrocytes with high expression of superficial zone protein (SZP) with low friction and joint lubrication as its functions. The transition, middle, and deep zones are characterized by prehypertrophic chondrocytes with high expression of glycosaminoglycans (GAG) and aggrecans (AGCs) with high water content to withstand the high compressive load exerted on the articular cartilage. The calcified zone is characterized by hypertrophic chondrocytes with high expression of collagen type X (Col X) and alkaline phosphatase (ALP) for the formation of a mineralized matrix for load transfer from the calcified zone to the underlying bone tissue. Therefore, injectable or implantable multilayer tissue constructs that simulate the zonal structure of articular cartilage would significantly improve the outcome and prevent permanent disability in patients suffering from PTOA.

Tissue-specific microcarriers for culture and expansion of cells, this approach could be used for culture and expansion of cells from all human tissues: articular cartilage microcarriers have been developed in Dr. Jabbari’s laboratory for culture and expansion of mesenchymal stem cells. Many potential uses exist for the current disclosure. -Cellular constructs for repair and regeneration of human tissues based on fused cell-seeded microcarriers with tissue-specific microcarriers and cells: Microgel-cell constructs have been developed in Dr. Jabbari’s laboratory for regeneration of articular cartilage tissue based on microcarriers from articular cartilage tissue and mesenchymal stem cells. -Multilayer cells constructs for simulating and mimicking the microstructure of human tissues: Multilayer microcell constructs have been developed in Dr. Jabbari’s laboratory for regeneration of zonal structure of articular cartilage with different layers simulating the superficial and calcified zone of articular cartilage. This approach can be used to simulate the microstructure of all human tissues from heart to skin, articular cartilage, blood vessels, nerve conduit, to ligament and tendon.
1) The use of microcarriers based on the ECM of the regenerating tissue allows direct use of microcarrier attached cells in tissue regeneration. This approach eliminates the need for detaching the cells from the microcarrier and seeding the cells in a scaffold for implantation, which is harmful to the cells and reduces cell viability and functionality.
2) The tissue-specific microcarriers in microcarrier-attached cells provide balanced cell-cell and cell-matrix interactions, mimicking the balance of cell-cell and cell-matrix interactions in the natural tissue being regenerated. This is not the case in cells encapsulated in hydrogels where cell-cell interaction is absent. This is also not the case in implantable cell pellets where cell-matrix interaction is absent. This is also not the case in implantable scaffolds where cell-cell contact is limited by insufficient pore interconnectivity. The tunable cell-cell and cell-matrix interactions in microcarrier-attached cells enhance our ability to regenerate multiple zones within a construct with each zone possessing a unique, optimum levels of cell-cell and cell-matrix interaction.
3) The use of tissue-specific microcarriers allows the production of multilayer constructs with each zone having zone-specific cell type and morphogens and zone-specific ECM, thus mimicking the structure of the natural tissue on the microscale.
4) The disclosed invention can be used for regeneration of any living tissue by generating microcarriers specific to the regenerating tissue from animal or human cadaver tissue. These living tissues include articular cartilage, bone, blood vessels, skin, heart, nerve, ligament, and tendon, among others.

The objective of the current disclosure is to develop a 3D microcarrier-based cell culture system that recreates the zonal structure of articular cartilage and can be implanted directly in an articular cartilage defect. To achieve the objective, fetal or adult bovine articular cartilage was decellularized and ground to form cartilage microparticles (CMPs) as three-dimensional substrates for expansion and delivery of human mesenchymal stem cells (MSCs). MSCs were expanded on fetal or adult CMPs in suspension culture to form fCMP-MSCs or aCMP-MSCs, respectively. The MSCs cultured on fetal or adult CMPs retained the expression of MSC markers. The MSCs, without detachment from CMPs, were used to form injectable hydrogels or implantable cells sheets for delivery to the site of articular cartilage defect.

For the injectable hydrogel, adult or fetal CMP-MSCs were suspended in alginate hydrogel, injected in a mold, cross-linked with calcium chloride, and cultured in chondrogenic medium.

For the implantable cell sheet, CMP-MSCs were suspended in culture medium, injected in a mold, allowed to settle gravitationally on the mold’s bottom surface, and incubated in chondrogenic medium for 48 h to form a monolayer cell sheet.

The previous steps were repeated to form a bilayer cell sheet consisting of fetal CMP-MSCs on top of adult CMP-MSCs. The injectable or implantable CMP-MSCs constructs were characterized with respect to cellularity, expression of chondrogenic markers, and compressive modulus. The injectable CMP-MSCs hydrogels (fetal or adult) had significantly higher expression of chondrogenic markers and compressive modulus after four (4) weeks incubation in chondrogenic medium compared to MSCs directly encapsulated in alginate hydrogel; implantable CMP-MSCs cell sheets had significantly higher expression of chondrogenic markers and compressive modulus compared to MSCs in the pellet culture. The implantable approach is potentially useful for creating multilayer cellular constructs by sequential settling of suspended CMP-MSCs in the medium to mimic the stratified structure of articular cartilage.

A promising alternative to chondrocyte harvesting is the use of “adult human mesenchymal stem cells”, hereafter referred to as MSCs. Based on previous studies, bone marrow and synovium derived MSCs have the highest chondrogenic potential compared to other sources. See, M. Amemiya, K. Tsuji, H. Katagiri, K. Miyatake, Y. Nakagawa, I. Sekiya, T. Muneta, H. Koga, Synovial fluid-derived mesenchymal cells have non-inferior chondrogenic potential and can be utilized for regenerative therapy as substitute for synovium-derived cells,Biochem Biophys Res Commun 523(2) (2020) 465-472 and X.B. Peng, Y. Zhang, Y.Q. Wang, Q. He, Q. Yu, IGF-1 and BMP-7 synergistically stimulate articular cartilage repairing in the rabbit knees by improving chondrogenic differentiation of bone-marrow mesenchymal stem cells, J Cell Biochem 120(4) (2019) 5570-5582. Although MSCs delivered in a supportive scaffold promote the expression of chondrogenic markers and produce a cartilage-like matrix in vivo, the approach of encapsulating MSCs in a uniform matrix, without gradients, leads to fibrocartilage formation and tissue degeneration. See, A.R. Armiento, M. Alini, M.J. Stoddart, Articular fibrocartilage - Why does hyaline cartilage fail to repair?, Adv Drug Deliv Rev 146 (2019) 289-305 and Y. Qi, Y. Du, W. Li, X. Dai, T. Zhao, W. Yan, Cartilage repair using mesenchymal stem cell (MSC) sheet and MSCs-loaded bilayer PLGA scaffold in a rabbit model, Knee Surgery, Sports Traumatology, Arthroscopy 22(6) (2014) 1424-1433. The formation of inferior fibrocartilage tissue is rooted in the inability of cellular tissue constructs to recapitulate the stratified structure of articular cartilage, that is, to provide a lubricating surface for the gliding joint motion and a loading-bearing matrix for interface with the underlying bone. See, D.F. Amanatullah, S. Yamane, A.H. Reddi, Distinct patterns of gene expression in the superficial, middle and deep zones of bovine articular cartilage, J Tissue Eng Regen Med 8(7) (2014) 505-14.. There is clearly a need for novel engineering approaches to recreate the zonal structure of full-thickness articular cartilage to inhibit the formation of fibrocartilage tissue.

The stratified structure of articular cartilage is composed of the superficial, middle, deep and calcified zones with each zone having a defined protein expression, cellularity, ECM composition and structure. See, S.P. Grogan, S.F. Duffy, C. Pauli, J.A. Koziol, A.I. Su, D.D. D’Lima, M.K. Lotz, Zone-specific gene expression patterns in articular cartilage, Arthritis Rheum 65(2) (2013) 418-28.

The superficial zone is populated with pre-chondrocytes expressing superficial zone protein (SZP) for joint lubrication and collagen types IX/II forming thin collagen fibrils. The middle/deep zones are characterized by pre-hypertrophic chondrocytes expressing aggrecan (AGC) and glycosaminoglycan (GAG) for compressive strength and collagen type II forming collagen fibrils thicker than those in the superficial zone. The calcified zone is populated with hypertrophic chondrocytes expressing alkaline phosphatase (ALP) and collagen type X, forming a mineralized matrix to interface with the subchondral bone.

It is well established that fetal articular cartilage, with a stratified structure from week 12 of gestation, has a higher regenerative capacity compared to the adult. See L. Wu, C. Bluguermann, L. Kyupelyan, B. Latour, S. Gonzalez, S. Shah, Z. Galic, S. Ge, Y. Zhu, F.A. Petrigliano, A. Nsair, S.G. Miriuka, X. Li, K.M. Lyons, G.M. Crooks, D.R. McAllister, B. Van Handel, J.S. Adams, D. Evseenko, Human developmental chondrogenesis as a basis for engineering chondrocytes from pluripotent stem cells, Stem Cell Reports 1(6) (2013) 575-89. This higher regenerative capacity is attributed to differences in cellularity and ECM composition between fetal and adult articular cartilage. The average cell density of fetal articular cartilage is 250 M cells/mL compared to 30 M cells/mL for the adult. The fraction of Col II in fetal articular cartilage is 77% of total collagen compared to 86% for the adult; the fraction of Col IX in fetal is 10% compared to 1% for the adult. The fetal articular cartilage has higher fraction of Col X compared to the fetal. See, A.G. Nerlich, T. Kirsch, I. Wiest, P. Betz, K. von der Mark, Localization of collagen X in human fetal and juvenile articular cartilage and bone, Histochemistry 98(5) (1992) 275-81.. Engineering approaches that mimic cellularity, ECM composition and structure of fetal articular cartilage could potentially accelerate regeneration of full-thickness articular cartilage defects.

We previously demonstrated for the first time that high cellularity, low matrix stiffness and combination of TGF-ß1 and BMP-7 led to chondrogenic differentiation of MSCs to the superficial zone phenotype of articular cartilage; medium cellularity and stiffness, and combination of TGF-ß1 and IGF-1 led to the middle zone phenotype; and low cellularity, high matrix stiffness and combination of TGF-ß1 and hydroxyapatite (HA) led to the calcified zone phenotype. See, T. Karimi, D. Barati, O. Karaman, S. Moeinzadeh, E. Jabbari, A developmentally inspired combined mechanical and biochemical signaling approach on zonal lineage commitment of mesenchymal stem cells in articular cartilage regeneration, Integrative Biology 7(1) (2014) 112-127 and S. Moeinzadeh, S.R.P. Shariati, E. Jabbari, Comparative effect of physicomechanical and biomolecular cues on zone-specific chondrogenic differentiation of mesenchymal stem cells, Biomaterials 92 (2016) 57-70. Further, we recently demonstrated that MSCs encapsulated in digested, decellularized articular cartilage can be sequentially differentiated to the superficial zone phenotype, followed by the middle and calcified zones of articular cartilage chondrocytes by sequential, timed supplementation with chondrogenic medium with BMP-7, IGF-1 and Indian hedgehog (IHH). S. Moeinzadeh, M. Monavarian, S. Kader, E. Jabbari, Sequential zonal chondrogenic differentiation of mesenchymal stem cells in cartilage matrices, Tissue Engineering Part A 25(3-4) (2019) 234-247. Although decellularized articular cartilage has been used as a matrix to stimulate chondrogenic differentiation of progenitor cells, see W.J. Vas, M. Shah, T.S. Blacker, M.R. Duchen, P. Sibbons, S.J. Roberts, Decellularized Cartilage Directs Chondrogenic Differentiation: Creation of a Fracture Callus Mimetic, Tissue Engineering Part A 24(17-18) (2018) 1364-1376, we showed for the first time that MSCs encapsulated in digested, decellularized, fetal bovine articular cartilage differentiated to the superficial zone phenotype of chondrocytes in chondrogenic medium whereas in the adult the MSCs differentiated to the calcified phenotype.

Owing to their large surface area, microcarriers like polystyrene or dextran microbeads are used in bioreactors for the expansion of MSCs. See C. Loubiere, C. Sion, N. De Isla, L. Reppel, E. Guedon, I. Chevalot, E. Olmos, Impact of the type of microcarrier and agitation modes on the expansion performances of mesenchymal stem cells derived from umbilical cord, Biotechnol Prog 35(6) (2019) e2887 and A.M. de Soure, A. Fernandes-Platzgummer, C.L. da Silva, J.M. Cabral, Scalable microcarrier-based manufacturing of mesenchymal stem/stromal cells, J Biotechnol 236 (2016) 88-109. Biodegradable microcarriers, based on poly(2-oxazoline) or alginate, have been developed as a 3D matrix for cultivation of stem cells and delivery to the site of regeneration. See S. Lück, R. Schubel, J. Rüb, D. Hahn, E. Mathieu, H. Zimmermann, D. Scharnweber, C. Werner, S. Pautot, R. Jordan, Tailored and biodegradable poly (2-oxazoline) microbeads as 3D matrices for stem cell culture in regenerative therapies, Biomaterials 79 (2016) 1-14 and S.I. Somo, K. Langert, C.-Y. Yang, M.K. Vaicik, V. Ibarra, A.A. Appel, B. Akar, M.-H. Cheng, E.M. Brey, Synthesis and evaluation of dual crosslinked alginate microbeads, Acta biomaterialia 65 (2018) 53-65. Commonly used microcarriers may cause undesirable phenotypic changes in MSCs. Further, an additional step is required to detach and separate the cells from the carrier for clinical applications.

The current disclosure provides a developmentally inspired, biomimetic microcarrier, based on decellularized articular cartilage, hereafter referred to as cartilage microparticles (CMPs), that will enhance the regenerative capacity of MSCs for articular cartilage defects. The MSCs adhered to CMPs, hereafter referred to as CMP-MSCs, can potentially mimic the process of fetal development of articular cartilage, in combination with zone-specific growth factors, following injection in the joint capsule using minimally invasive arthroscopic techniques. See, T.H. Yoon, M. Jung, C.H. Choi, H.S. Kim, Y.H. Lee, Y.S. Choi, S.J. Kim, S.H. Kim, Arthroscopic gel-type autologous chondrocyte implantation presents histologic evidence of regenerating hyaline-like cartilage in the knee with articular cartilage defect, Knee Surg Sports Traumatol Arthrosc 28(3) (2020) 941-951. Further, CMP-MSCs can be used to assemble multilayer constructs with gradients in cell density, matrix composition, and morphogens to stimulate regeneration of zonal structure of articular cartilage.

The following approach was used to test the hypothesis that CMP-MCs enhance chondrogenesis for articular cartilage regeneration, as illustrated schematically in FIG. 1. Fetal or adult bovine articular cartilage was decellularized, frozen in liquid nitrogen and milled. Next, the freeze-dried fragments were ground and sorted by passing through sieves to generate fetal or adult CMP fractions with average sizes of 90, 190 and 250 µm. The fetal and adult CMPs are hereafter referred to as fCMPs and aCMPs, respectively. The CMPs were characterized by size distribution, water content, and mass loss. The fCMPs and aCMPs were used as a microcarrier for growth and expansion of MSCs to generate fCMP-MSCs and aCMP-MSCs, respectively. Next, two approaches were used to deliver the CMP-MSCs to the site of articular cartilage regeneration, namely an injectable hydrogel approach and a prefabricated hydrogel approach. In the injectable hydrogel approach, fCMP-MSCs or aCMP-MSCs with different average sizes were encapsulated in alginate hydrogels and cultured in chondrogenic medium. The CMP-MSCs encapsulated in alginate hydrogel is hereafter referred to as CMP-MSCs/alg. In the prefabricated hydrogel approach, CMP-MSCs were suspended in chondrogenic medium, allowed to settle on bottom surface of the culture plate by gravitational force, the settled CMP-MSCs were incubated in chondrogenic medium for 48 h to form a continuous cell monolayer sheet. Bilayer cell sheets was generated by gravitational settling of aCMP-MSCs and incubation to form a continuous cell sheet followed by gravitational settling of fCMP-MSCs on top of the fetal monolayer sheet and incubation to form a fetal-adult bilayer. The monolayer and bilayer CMP-MSCs cell sheets are hereafter referred to as CMP-MSCs/ml and CMP-MSCs/bl, respectively. The CMP-MSCs/alg, CMP-MSCs/ml and CMP-MSCs/bl were incubated in chondrogenic medium and assessed with respect to compressive modulus and expression of chondrogenic markers of the superficial zone (Sox-9 and SZP), middle zone (Col II and AGC), and calcified zone (Col X and ALP). Results demonstrate that CMPs, as a 3D substrate for adhesion of MSCs, increase compressive modulus and expression of chondrogenic markers of MSCs in injectable alginates or implantable cell sheets, as compared to direct encapsulation of MSCs in alginates or MSC pellets, respectively.

### Materials and Methods

### Materials

Paraformaldehyde, formalin, paraffin, ethylenediaminetetraacetic acid (EDTA), penicillin G, streptomycin and tris(hydroxymethyl)aminomethane (Tris) were received from Sigma-Aldrich (St. Louis, MO). The sodium salt of pyruvic acid, L-proline, DNase I and RNase A were received from Amresco (Dublin, Ireland). Sodium alginate was received from Ward’s Science (Henrietta, NY). Iodoacetic acid and calcium chloride were received from ThermoFisher Scientific (Rockford, IL). Dichloromethane (DCM, Acros Organics, Pittsburg, PA) was dried by distillation over calcium hydride. Diethyl ether, dimethylformamide (DMF) and hexane were received from VWR (Bristol, CT) and used as received. Polyester filter sieves with 80, 90, 180, 190, 240 and 250 µm pore size were obtained from Rosin Tech (Los Angeles, CA). Nylon cell strainers with 40 and 70 µm sizes were received from Corning (Canton, NY). Polystyrene microparticles with 50 µm diameter and no surface modification were received from Advance Scientific (Moffat Beach, QLD, Australia).

Full-thickness adult and fetal articular cartilage harvested from the bovine femoral condyles were received from Animal Technologies (Tyler, TX). Adult human mesenchymal stem cells, hereafter referred to as MSCs, harvested from healthy human bone marrow with high expression of CD105, CD166, CD29, and CD44 and low expression of CD14, CD34 and CD45 markers and TGF-ß1 were received from Lonza (Allendale, NJ). Dulbecco’s modified eagle medium (DMEM) cell culture medium, Dulbecco’s phosphate-buffer saline (PBS), fetal bovine serum (FBS), trypsin-EDTA, Quant-it PicoGreen dsDNA reagent kit, and the live/dead staining kit, consisting of acetomethoxy derivative of calcein (cAM) and ethidium homodimer (EthD) were received from Life Technologies (Grand Island, NY). QuantiChrom alkaline phosphatase (ALP) assay kit was received from Bioassay Systems (Hayward, CA). 1,9-dimethylmethylene blue (DMMB) assay kit for quantification of glycosaminoglycan (GAG) content, hematoxylin and eosin-Y (H&E) for staining cell nuclei and cytoplasm, and Alcian blue for GAG staining were received from Sigma-Aldrich. PicoGreen assay kit for quantification of double-stranded DNA content was received from Molecular Probes (ThermoFisher Scientific, Waltham, MA). All primary and secondary antibodies were received from Santa Cruz Biotechnology (Dallas, TX). All forward and reverse primers were synthesized and received from Integrated DNA Technologies (Coralville, IA).

### Production of Decellularized Bovine Cartilage Microparticles

Full thickness articular cartilage samples, harvested from fetal or adult bovine femoral condyles, were decellularized as we described previously. Briefly, the articular cartilage samples were dissected with a scalpel into 5 × 5 × 2 mm pieces, the dissected pieces were frozen in liquid nitrogen and milled. The milled fragments were decellularized by immersion in 10 Mm Tris/1% triton solution for 24 h followed by sonication for 2 h at 55 kHz. Next, the sonicated fragments were immersed in nuclease solution consisting of 1 U/mL deoxyribonuclease and 1 U/mL ribonuclease in PBS for 72 h at 37° C. to degrade DNA and RNA. See T.W. Gilbert, T.L. Sellaro, S.F. Badylak, Decellularization of tissues and organs, Biomaterials 27(19) (2006) 3675-3683. The decellularized fragments were washed 3X in PBS, centrifuged, the supernatant was discarded, and the solid was freeze-dried.

The freeze-dried fragments were further grinded (Hamilton Beach, Southern Pines, NC) and sorted for size by progressively passing through sieves ranging in size from 80 to 300 µm. First, the soft fragments were passed through 80 and 300 µm sieves to eliminate sizes <80 µm and >300 µm. Next, the soft, fetal or adult decellularized cartilage microparticles, hereafter referred to as CMPs, were passed through a 90 µm sieve to collect a fraction with 40-110 µm size range, which is referred to as the 90 µm CMPs. Next, the >90 µm CMPs were passed through a 190 µm sieve to collect a fraction with 60-220 µm size range, which is referred to as the 190 µm CMPs. Then, the >190 µm CMPs were passed through a 250 µm sieves to collect a fraction with 60-300 µm size range, which is referred to as the 250 µm CMPs. The >250 µm fraction was further grinded and the above process was repeated to divide the particles into three fractions with average sizes of 90, 190 and 250 µm. Fetal and adult CMPs are hereafter referred to as fCMPs and aCMPs, respectively.

### Characterization of the Decellularized Articular Cartilage Microparticles

Microparticle size distribution: The sieved CMPs were imaged with a light microscope to determine their size distribution. The captured 2D images were analyzed with ImageJ software (National Institutes of Health, Bethesda, MD) to determine the average size as we described previously. See O. Karaman, A. Kumar, S. Moeinzadeh, X. He, T. Cui, E. Jabbari, Effect of surface modification of nanofibres with glutamic acid peptide on calcium phosphate nucleation and osteogenic differentiation of marrow stromal cells, J Tissue Eng Regen Med 10(2) (2016) E132-46. After size analysis, the three CMP size fractions, from fetal and adult bovine articular cartilages, were immersed in liquid nitrogen and cut with a surgical blade to expose a freshly cut surface for morphological analysis. Next, the CMPs were coated with gold using a Denton Desk II sputter coater (Moorestown, NJ) at 20 mA for 75 s. The CMPs were imaged for morphological analysis with a TESCAN VEGA3 SBU variable-pressure scanning electron microscope (SEM; Kohoutovice, Czech Republic) at an accelerating voltage of 8 keV.

Measurements of water content and mass loss: The equilibrium water content of dried, decellularized CMPs was measured by incubation in phosphate buffer saline (PBS) at 37° C. as we described previously. See, D. Barati, S. Kader, S.R. Pajoum Shariati, S. Moeinzadeh, R.H. Sawyer, E. Jabbari, Synthesis and Characterization of Photo-Cross-Linkable Keratin Hydrogels for Stem Cell Encapsulation, Biomacromolecules 18(2) (2017) 398-412. Briefly after swelling in PBS, the microparticles were filtered, unbound water was removed with a filter paper, and the sample weight was measured. After weighing, the sample was returned to a fresh solution and incubated until the next time point. This process was repeated until equilibrium or maximum swelling was achieved. The water content of CMPs at equilibrium was calculated as the difference between the initial and swollen weight divided by the total weight of the CMPs. The mass loss of CMPs samples was measured at one time point after 8 weeks of incubation in PBS. After 8 weeks, the CMPs were filtered, freeze-dried, and the sample weight was measured. Mass loss was defined as the difference between the initial and final weight of the dry CMPs divided by the initial dry weight.

### Culture of MSCs on Articular Cartilage Microparticles

MSCs (passage 3-5) were expanded in a high glucose DMEM medium supplemented with 10% FBS, 100 units/mL penicillin G, and 100 µg/mL streptomycin (basal medium, BM) as we previously described. See S. Moeinzadeh, M. Monavarian, S. Kader, E. Jabbari, Sequential zonal chondrogenic differentiation of mesenchymal stem cells in cartilage matrices, Tissue Engineering Part A 25(3-4) (2019) 234-247. CMPs were sterilized by immersion in ethanol for 2 h followed by exposure to ultraviolet (UV) radiation for 30 min. See Y.B. Kim, G.H. Kim, PCL/alginate composite scaffolds for hard tissue engineering: fabrication, characterization, and cellular activities, ACS combinatorial science 17(2) (2015) 87-99. The sterilized fCMPs or aCMPs were incubated in basal culture medium overnight to reach equilibrium swelling prior to cell seeding. The initial seeding density was calculated based on the typical average size of MSCs (18 µm average diameter, see L. Liu, L. Tseng, Q. Ye, Y.L. Wu, D.J. Bain, C. Ho, A New Method for Preparing Mesenchymal Stem Cells and Labeling with Ferumoxytol for Cell Tracking by MRI, Sci Rep 6 (2016) 26271.) and the size and surface area of CMPs. The initial cell seeding density was based on 1 × 10⁶ MSCs occupying 20% of surface area of the CMPs (20% initial confluency). The volume porosity of CMPs was measured from the percent mass swellings assuming the CMP density did not change significantly with swelling. The CMPs were characterized by an average size equivalent to the diameter of a sphere and the CMP pores were assumed to be cubical in shape. The porosity, pore volume, surface area, and the number and mass of CMPs required for initial 1 × 10⁶ MSC seeding to occupy 20% of the surface area for fCMPs and aCMPs with 90, 190 and 250 µm sizes are given in Table 1. For fCMPs or aCMPs with average sizes of 90, 190 and 250 µm, the amounts of microparticles required for 3 × 10⁶ cells were 0.22, 0.48 and 0.64 mg, respectively. For 3D microcarrier cell culture, the specified amount of CMPs were added to the basal medium in ultra-low attachment T-75 tissue culture flasks followed by the addition of 3 × 10⁶ MSCs. The flasks were securely mounted on a rocker mixer and placed in a humidified 5% CO₂ incubator at 37° C. A doubling time of 48 h is reported for MSCs cultured on biological extracellular matrices (ECM). See P.J. Dolley-Sonneville, L.E. Romeo, Z.K. Melkoumian, Synthetic surface for expansion of human mesenchymal stem cells in xeno-free, chemically defined culture conditions, PLoS One 8(8) (2013) e70263. Based on this doubling time, fresh CMPs were added to the cell culture medium every 48 h to limit confluency to 40% and prevent cell crowding on the surface of CMPs. The analysis of live and dead images of the grown MSCs confirmed the absence of cell crowding on the surface and in the pore volume of the CMPs. MSCs cultured on 2D adherent culture flasks and MSCs cultured on 3D polystyrene microbeads (50 µm average size) were used as controls. At each time point (7, 14 and 21 days), the cultures were characterized with respect to cell number and viability, and mRNA expression of MSC markers.

### Characterization of MSCs Cultured on Articular Cartilage Microparticles

Cell viability and growth: At each time point (7, 14 and 21 days), 2 mL of 0.05% trypsin/ 0.53 mM EDTA was added to 1 mL of the cell culture suspension and incubated for 15 min under shaking to detach MSCs from the CMPs. Next, the suspension was transferred on a 40 µm nylon cell strainer fixed on a 50 mL Falcon tube and washed with DMEM using an insulin syringe. The filtrate was centrifuged at 400 × g for 5 min as we described previously. See X. He, E. Jabbari, Material properties and cytocompatibility of injectable MMP degradable poly(lactide ethylene oxide fumarate) hydrogel as a carrier for marrow stromal cells, Biomacromolecules 8(3) (2007) 780-92, and the separated cells were counted with a hemocytometer. For cell viability, the CMPs were incubated with 1 µg/mL cAM/EthD to stain live and dead cells, respectively, as we described previously. See D. Barati, S. Kader, S.R. Pajoum Shariati, S. Moeinzadeh, R.H. Sawyer, E. Jabbari, Synthesis and Characterization of Photo-Cross-Linkable Keratin Hydrogels for Stem Cell Encapsulation, Biomacromolecules 18(2) (2017) 398-412. The stained cells were imaged using an inverted fluorescent microscope (Nikon Eclipse Ti-e, Nikon, Melville, NY).

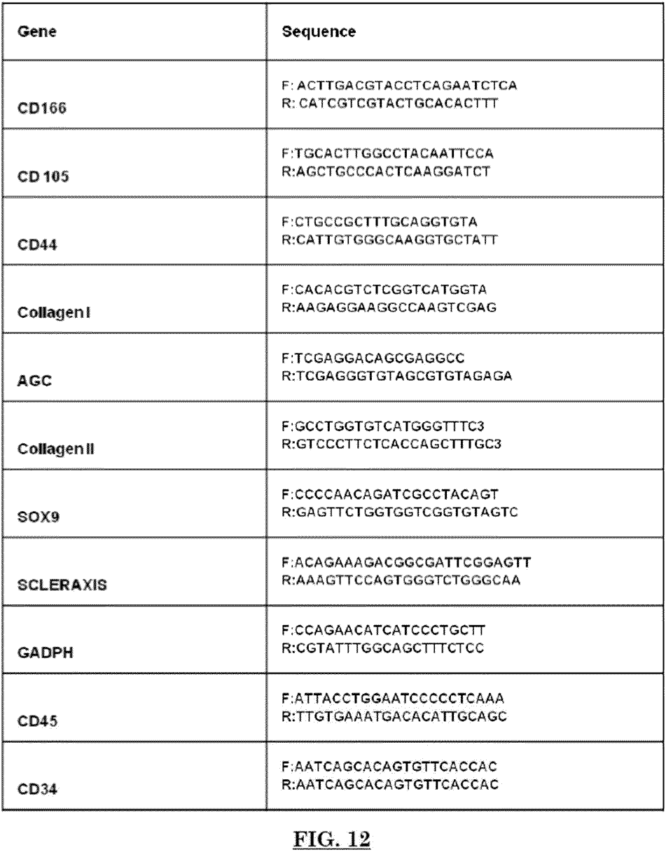

Analysis of mRNA expression: The mRNA expression of CD105, CD166 and CD44 markers are up-regulated in MSCs whereas those of CD45 and CD34 are downregulated. See M.A. Crabbé, K. Gijbels, A. Visser, D. Craeye, S. Walbers, J. Pinxteren, R.J. Deans, W. Annaert, B.L. Vaes, Using miRNA-mRNA Interaction Analysis to Link Biologically Relevant miRNAs to Stem Cell Identity Testing for Next-Generation Culturing Development, Stem Cells Translational Medicine 5(6) (2016) 709-722. Therefore, MSCs cultured on CMPs were characterized phenotypically by their mRNA expression of CD105, CD166, CD44, CD45 and CD34 markers. Further, the CMP cultured MSCs were tested for differentiation to the chondrogenic lineage by measuring the mRNA expression of chondrogenic markers, SOX-9, Collagen I (Col I), Collagen II (Col II), and aggrecan (AGC), see S. Moeinzadeh, S.R.P. Shariati, E. Jabbari, Comparative effect of physicomechanical and biomolecular cues on zone-specific chondrogenic differentiation of mesenchymal stem cells, Biomaterials 92 (2016) 57-70 and S. Moeinzadeh, M. Monavarian, S. Kader, E. Jabbari, Sequential zonal chondrogenic differentiation of mesenchymal stem cells in cartilage matrices, Tissue Engineering Part A 25(3-4) (2019) 234-247. At each time point, MSCs were separated from the CMPs as described above and the total RNA of the homogenized cell suspension was isolated using TRIzol as we described previously. See, O. Karaman, A. Kumar, S. Moeinzadeh, X. He, T. Cui, E. Jabbari, Effect of surface modification of nanofibres with glutamic acid peptide on calcium phosphate nucleation and osteogenic differentiation of marrow stromal cells, J Tissue Eng Regen Med 10(2) (2016) E132-46. The genomic DNA was removed using deoxyribonuclease I (Invitrogen) as previously described. See, T. Karimi, D. Barati, O. Karaman, S. Moeinzadeh, E. Jabbari, A developmentally inspired combined mechanical and biochemical signaling approach on zonal lineage commitment of mesenchymal stem cells in articular cartilage regeneration, Integrative Biology 7(1) (2014) 112-127. 250 ng of the extracted RNA, as measured using a Nanodrop spectrophotometer, was converted to cDNA using Promega reverse transcription system (Madison, WI). The cDNA was amplified with Eppendorf SYBR green RealMasterMix (Hamburg, Germany) using a Bio-Rad CXF96 real-time quantitative polymerase chain reaction system (rt-qPCR; Hercules, CA) and the appropriate gene-specific primers as descried. See, S. Moeinzadeh, S.R.P. Shariati, E. Jabbari, Comparative effect of physicomechanical and biomolecular cues on zone-specific chondrogenic differentiation of mesenchymal stem cells, Biomaterials 92 (2016) 57-70. The primer sequences, as listed in Table 1, see FIG. 12, were designed and selected using Primer3 web-based software as described, see T. Karimi, D. Barati, O. Karaman, S. Moeinzadeh, E. Jabbari, A developmentally inspired combined mechanical and biochemical signaling approach on zonal lineage commitment of mesenchymal stem cells in articular cartilage regeneration, Integrative Biology 7(1) (2014) 112-127. The expressions were normalized against GAPDH reference gene and fold changes were compared based on ΔΔct method to those in the same group at day zero as previously described. K.W. Kavalkovich, R.E. Boynton, J.M. Murphy, F. Barry, Chondrogenic differentiation of human mesenchymal stem cells within an alginate layer culture system, In Vitro Cellular & Developmental Biology-Animal 38(8) (2002) 457-466.

### Encapsulation of CMP-MSCs in Alginate as an Injectable Hydrogel

CMP-MSCs were encapsulated in sodium alginate hydrogel cross-linked with calcium chloride (CaCl2) using the following procedure. See, K.Y. Lee, D.J. Mooney, Alginate: properties and biomedical applications, Prog Polym Sci 37(1) (2012) 106-126. The sodium alginate solution was prepared by adding 3 g sodium alginate to 100 mL PBS under stirring. The CaCl2 solution was prepared by adding 1 g CaCl₂ to 100 mL PBS under stirring. The alginate and CaCl₂ solutions were sterilized by filtration. A suspension of adult or fetal CMP-MSCs in culture medium with average CMP sizes of 90, 190, and 250 µm was prepared as described herein. The suspension was transferred to a sterilized 15 mL Falcon tube, centrifuged and the medium was removed. Next, the alginate solution was added to the CMP-MSCs and mixed with a pre-sterilized glass rod. Then, the surface of a pre-sterilized, disk-shaped, Teflon mold with effective diameter of 2 cm and height of 1.5 mm was sprayed with the 1% CaCl₂ solution using an insulin syringe. Next, the suspension of CMP-MSCs in sodium alginate was transferred to the mold and CaCl₂ solution was sprayed on the exposed surface to crosslink the suspension. The gelation time was determined visually by placing a drop of the alginate solution on a glass ruler, followed by spraying CaCl₂ solution on the drop, tilting the ruler by 45 degrees, observing movement of the drop on the titled ruler, and recording the time when the drop stopped moving as the gelation time. The crosslinking time of the CMP-MSC encapsulated alginate hydrogels ranged from 1-4 min. After crosslinking, the molds were transferred to a petri dish and cultured in chondrogenic medium for up to 8 weeks. The final concentration of alginate in the hydrogels was 3% w/v. MSCs encapsulated directly in the alginate gel, without CMPs, at a density of 1 × 10⁵ cells/mL were used as the control group. aCMP-MSCs and fCMP-MSCs encapsulated in alginate gels is hereafter referred to as aCMP-MSCs/alg and fCMP-MSCs/alg, respectively.

### Formation of CMP-MSC Monolayers and Bilayers as a Solid Implant

The following approach was used to generate MSC cell sheets for implantation in an articular cartilage defect, as opposed to injection and in situ gelation. This approach is similar to MSC pellet cultures, see S. Giovannini, J. Diaz-Romero, T. Aigner, P. Heini, P. Mainil-Varlet, D. Nesic, Micromass co-culture of human articular chondrocytes and human bone marrow mesenchymal stem cells to investigate stable neocartilage tissue formation in vitro, Eur Cell Mater 20(245) (2010) 59., but adapted to CMP-MSCs. MSCs were cultured in 3D on adult/fetal CMPs, with average sizes of 90, 190, and 250 µm, to approximately 50% confluency to form adult/fetal CMP-MSCs, as described herein. Next, a suspension of adult/fetal CMP-MSCs in culture medium was transferred to a sterile Teflon mold (1.55 mm depth and 2 cm in diameter), the mold was placed in a sterile petri dish, and the assembly was incubated in a humidified 5% CO2 incubator at 37° C. for 48 h. During incubation, gravitational settling of aCMP-MSCs or fCMP-MSCs on bottom surface of the mold and secretion of extracellular matrix (ECM) led to the formation of an aCMP-MSCs or fCMP-MSCs monolayer cell sheet, hereafter referred to as aCMP-MSCs/ml and fCMP-MSCs/ml cell sheet, respectively. The CMP-MSC loading in the culture medium was adjusted to form a 0.75 mm thick cell sheet. To mimic the zonal structure of articular cartilage, the following approach was used to produce bilayer cell sheets. After formation of aCMP-MSC/ml, a suspension of fCMP-MSCs was transferred to the aCMP-MSC/ml mold, and the assembly was incubated for another 48 h. During incubation, gravitational settling of fCMP-MSCs on aCMP-MSCs/ml on bottom surface of the mold and secretion of extracellular matrix (ECM) led to the formation a bilayer cell sheet composed of fCMP-MSCs/ml on top of an aCMP-MSCs/ml, hereafter referred to as faCMP-MSCs/bl cell sheet. The CMP-MSC loading in the culture medium was adjusted to form a 1.5 mm faCMP-MSCs/bl cell sheet. After cell sheet formation, the medium was replaced with chondrogenic medium and the cell sheets were cultured for up to 8 weeks. The chondrogenic medium consisted of DMEM (4.5 g/mL glucose, 50 µg/ mL L-proline, 50 µg/mL ascorbic acid, 0.1 mM sodium pyruvate, 1% v/v insulin-transferrin-selenium premix) supplemented with the 10 ng/mL TGF-ß1. MSC pellets formed directly by centrifugation from suspension were used as the control group. commonly used pellet cultures. H. Rogan, F. Ilagan, F. Yang, Comparing Single Cell Versus Pellet Encapsulation of Mesenchymal Stem Cells in Three-Dimensional Hydrogels for Cartilage Regeneration, Tissue Eng Part A 25(19-20) (2019) 1404-1412.

### Analysis of Monolayer, Bilayer, or Alginate Encapsulated CMP-MSCs

At each time point, adult or fetal CMP-MSCs/alg, CMP-MSCs/ml and CMP-MSCs/bl were assessed with respect to compressive modulus, cellularity, and the expression of chondrogenic markers Sox-9, Collagen II (Col II) and aggrecan (AGC), the superficial zone marker SZP and calcified zone markers collagen X (Col X) and alkaline phosphatase (ALP). Cell viability was assessed by imaging with live & dead cell assay. The mono/bi-layer sheets were incubated with acetyl methoxy derivative of calcein (cAM) and ethidium homodimer (EthD) as live & dead cell stains, respectively, as we described previously, and the stained samples were imaged using the Eclipse Ti-E inverted fluorescent microscope. The CMP-MSCs/alg gels were rinsed twice with PBS and fixed with 4% paraformaldehyde for 3 h. After fixation, cells were permeabilized using PBS containing 0.1% Triton X-100 for 5 min, rinsed, and incubated with Alexa 488 phalloidin (1:200 dilution) and DAPI (1:5000 dilution) to stain actin filaments of the cell cytoskeleton and cell nuclei, respectively, as previously described. See, K. Yang, J. Sun, D. Wei, L. Yuan, J. Yang, L. Guo, H. Fan, X. Zhang, Photo-crosslinked mono-component type II collagen hydrogel as a matrix to induce chondrogenic differentiation of bone marrow mesenchymal stem cells, Journal of Materials Chemistry B 5(44) (2017) 8707-8718. The stained hydrogels were imaged with the Nikon inverted fluorescent microscope. At each time point, the samples (CMP-MSCs/alg, CMP-MSCs/ml, or CMP-MSCs/bl) were homogenized and the mRNA expression of chondrogenic markers Sox-9, Col II, Col X, SZP, AGC and ALP were measured as described herein.

### Histological Analysis

The aCMP-MSCs/ml, fCMP-MSCs/ml, or faCMP-MSCs/bl, after 21 days of culture, were analyzed histologically for the expression of GAG and mineralized deposits as previously described, see T. Karimi, D. Barati, O. Karaman, S. Moeinzadeh, E. Jabbari, A developmentally inspired combined mechanical and biochemical signaling approach on zonal lineage commitment of mesenchymal stem cells in articular cartilage regeneration, Integrative Biology 7(1) (2014) 112-127. Briefly, the samples were fixed in formalin, embedded in paraffin, and cryo-sectioned to a thickness of 10 µm. The sections were divided into three groups with the first group stained with H&E to ascertain morphology of the encapsulated cells, Alcian blue to image GAG accumulation, and Alizarin red to image mineral deposits. The stained sections were imaged with a Nikon Optiphot Epi-florescence microscope.

### Compressive Modulus of Monolayer, Bilayer, or Alginate Encapsulated CMP-MSCs

At each time point, adult or fetal CMP-MSCs/alg, CMP-MSCs/ml, or CMP-MSCs/bl were loaded on the Peltier plate of an AR 2000ex rheometer (TA Instruments, New Castle, DE, USA) and subjected to a uniaxial compressive strain as we previously described. See, S. Moeinzadeh, D. Barati, X. He, E. Jabbari, Gelation characteristics and osteogenic differentiation of stromal cells in inert hydrolytically degradable micellar polyethylene glycol hydrogels, Biomacromolecules 13(7) (2012) 2073-86. A strain sweep from 0.01% to 500% strain at 10 Hz was performed to find the yield strain. Similarly, a frequency sweep from 0.01 to 100 Hz at 0.2% strain was done to find the crossover frequency. A sinusoidal shear strain with a frequency above the crossover frequency and a strain amplitude below the yield strain was exerted on the sample and the storage (G‘) and loss moduli (G“) were recorded with time. The slope of the linear fit to the stress-strain curve for strains of <10% was taken as the compressive modulus of the gel sample.

### Statistical Analysis

All experiments were done in triplicate and quantitative data is expressed as means + standard deviation. Significant differences between groups were evaluated using a two-way ANOVA with replication test and two-tailed Student’s t-tests. A value of p > 0.05 was considered statistically significant.

### Results

FIG. 2 at a-c shows size distribution of aCMPs with average sizes of 90 µm, 190 µm and 250 µm, respectively; FIG. 2 at d-f show size distribution of fCMPs with average sizes of 90 µm, 190 µm and 250 µm; the insets in FIG. 2 at a-f show the corresponding microscope images of the CMPs. For fetal as well as adult CMPs, the fractions corresponding to the average size was between 70-90% of the distribution for the three samples. The fraction of particles with size greater than the average was higher for fCMPs compared to the aCMPs, which was attributed to the softer texture of fCMPs, and led to their passage through the sieve with slight pressure. The harder texture of aCMPs resulted in a smaller particle size distribution compared to fetal. The CMPs had irregular, non-spherical shapes, as shown in the inset of FIG. 2 at a-f. There was no difference in the shape of fetal and adult CMPs.

The percent equilibrium water contents of the aCMPs with average particle size of 90 µm, 190 µm and 250 µm were 18.6±0.9%, 19.6±0.9% and 20.0±1.5%, respectively; the water content of aCMPs were 15.3±1.5%, 16.5±0.6% and 18.0±0.8%. The water content of fCMPs was not affected by their particle size whereas the water content of aCMPs increased slightly with increasing size. For all particle sizes, the water content of fCMPs was significantly higher than aCMPs. The mass loss of fCMPs with average particle size of 90 µm, 190 µm and 250 µm after 8 weeks incubation in PBS was 4.4±0.2%, 5.2±0.2% and 6.5±0.1%, respectively; the mass loss of aCMPs was 3.7±0.3%, 4.3±1.3% and 5.8±0.5%. The mass loss of fetal and adult CMPs significantly increased with increasing particle size. For a given particle size, the mass loss of aCMPs was slightly lower than fCMPs. The mass loss data indicated that the fetal and adult CMPs are stable for up to 8 weeks with negligible hydrolytic degradation in the absence of enzymes.

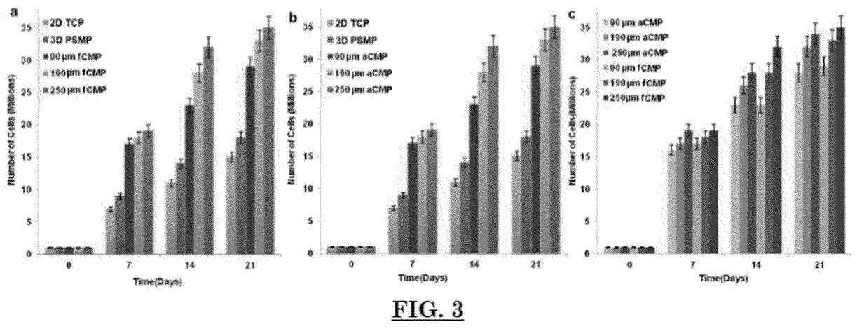

Two culture methods were tested for expansion of MSCs on CMPs. In the first method, all CMPs were dispersed in basal culture medium along with MSCs initially at time zero. In the second method, all MSCs were added to the culture medium initially at time zero but the CMPs were added gradually every two days starting at zero time. The second method was more efficient as the surface area for cell adhesion and growth was increased gradually without allowing the percent cell confluency to fall below 20%, which improved cell-cell communication between the adhered MSCs. The second method allowed continuous expansion of MSCs by addition of more CMPs to the culture medium with incubation time. FIG. 3 at a and b compare the cell content of fetal and adult CMPs, respectively, with average sizes of 90 µm, 190 µm and 250 µm with incubation time against MSCs grown on 3D polystyrene microparticles (PSMP). For all time points, the cell content of the CMP groups was significantly higher than the control and increased with incubation time for all CMP sizes. For all time points, the CMPs with 250 µm average size had highest cell content compared to other CMP sizes for fetal as well as adult CMPs. The lower cell content of the 90 µm CMPs compared to the 250 µm CMPs can be attributed to the higher inter-particle cell transfer between the CMPs with each successive CMP addition to the culture medium. Inter-particle transfer requires cell detachment from one particle, migration through the medium, and re-attachment to another particle with lower cell content, which increased the lag time between cell divisions. FIG. 3 at c compares the cell content of fetal and adult CMPs as a function of average particle size with incubation time. For a given time and particle size, the cell content of fCMPs was slightly higher than aCMPs but the difference was not statistically significant.

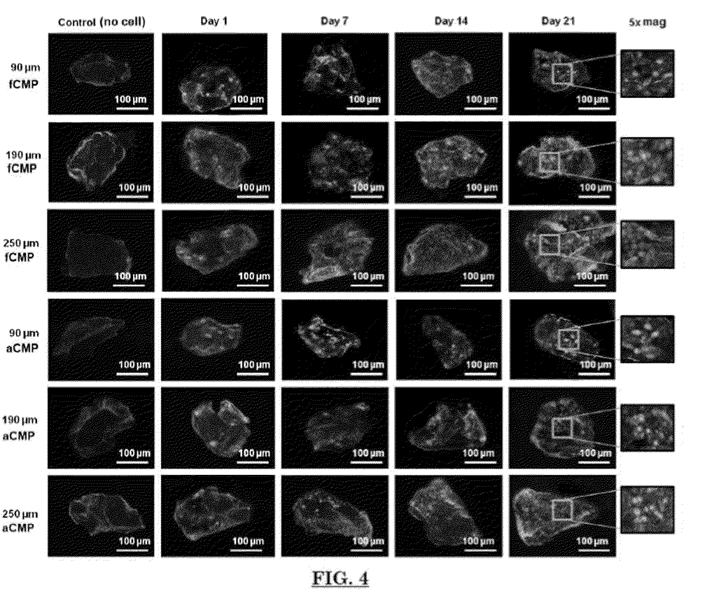

The live (green) and dead (red) fluorescent images of a randomly selected particle from the CMP samples as a function of incubation time are shown in FIG. 4 for fetal as well as adult CMPs of different average sizes. The fluorescent images showed the presence of cells in the porous structure of CMPs, showing that the pores were sufficiently large for cell penetration within the CMPs. The intensity of green fluorescence from the CMPs increased with incubation time for fetal as well as adult CMPs and for all particle sizes. After 21 days of incubation, the fluorescent intensity of fCMPs was slightly higher than aCMPs. However, no significant difference was observed between the fluorescent intensities of CMPs with different average sizes. The fluorescent images showed >95% cell viability for CMP-MSCs.

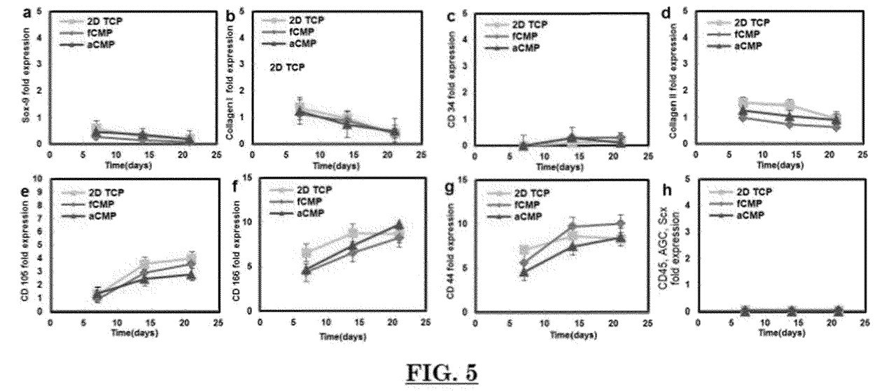

Based on previous studies, MSCs harvested from the human bone marrow express CD105 and CD44 markers whereas the expression of CD45 and CD34 markers is down regulated. See M.C. Kastrinaki, I. Andreakou, P. Charbord, H.A. Papadaki, Isolation of human bone marrow mesenchymal stem cells using different membrane markers: comparison of colony/cloning efficiency, differentiation potential, and molecular profile, Tissue Eng Part C Methods 14(4) (2008) 333-9 and A.Y. Lee, J. Lee, C.L. Kim, K.S. Lee, S.H. Lee, N.Y. Gu, J.M. Kim, B.C. Lee, O.J. Koo, J.Y. Song, S.H. Cha, Comparative studies on proliferation, molecular markers and differentiation potential of mesenchymal stem cells from various tissues (adipose, bone marrow, ear skin, abdominal skin, and lung) and maintenance of multipotency during serial passages in miniature pig, Res Vet Sci 100 (2015) 115-24. Further, the as-received MSCs had high expression of CD105, CD166, CD29, and CD44 and low expression of CD14, CD34 and CD45 markers. Therefore, the MSCs expanded on fetal/adult CMPs were evaluated with respect to retention of the expression of CD 105, CD166 an CD44 markers and the absence of CD45 and CD34 expression, see FIG. 5. As shown in FIG. 5, the expression of CD105, CD166 and CD44 markers of MSCs expanded on fetal/adult CMPs in basal MSC medium increased with incubation time whereas CD45 and CD34 markers were not expressed. There was no significant difference in the marker expression between CMP-MSCs versus MSCs culture on 2D plates; there was also no significant difference in marker expression between aCMP-MSCs and fCMP-MSCs. The decellularized cartilage microparticles could prematurely induce differentiation of MSCs during the expansion phase. Therefore, the expression of chondrogenic and osteogenic markers were measured in addition to MSC markers. These included Sox-9 as the master regulator of chondrogenesis, Col II as the chondrogenic marker, and Col I as the osteogenic marker, FIG. 5. See L. Gao, T.J. Sheu, Y. Dong, D.M. Hoak, M.J. Zuscik, E.M. Schwarz, M.J. Hilton, R.J. O’Keefe, J.H. Jonason, TAK1 regulates SOX9 expression in chondrocytes and is essential for postnatal development of the growth plate and articular cartilages, J Cell Sci 126(Pt 24) (2013) 5704-13. According to FIG. 5, the expression of Sox-9, Col II and Col I of CMP-MSCs decreased with incubation time in basal medium. Further, there was no difference in the expression of chondrogenic markers of fCMP-MSCs and aCMP-MSCs.

The results in FIG. 5 demonstrate that MSCs can be expanded on fetal or adult CMPs in basal medium without premature differentiation.

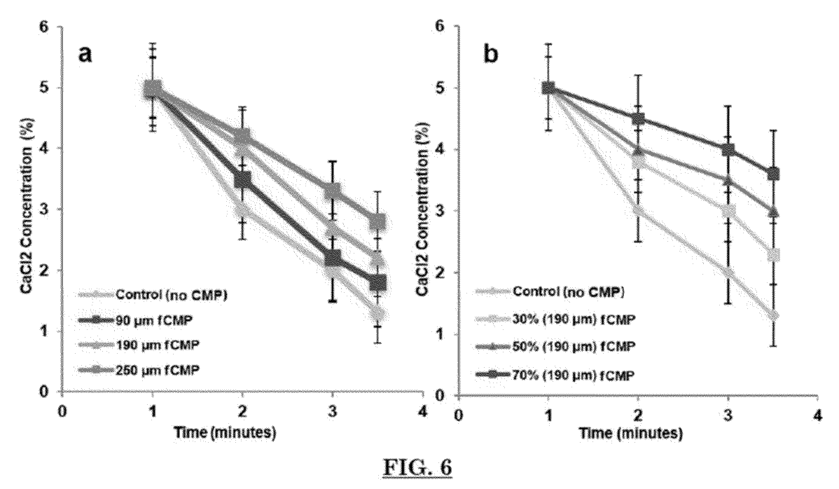

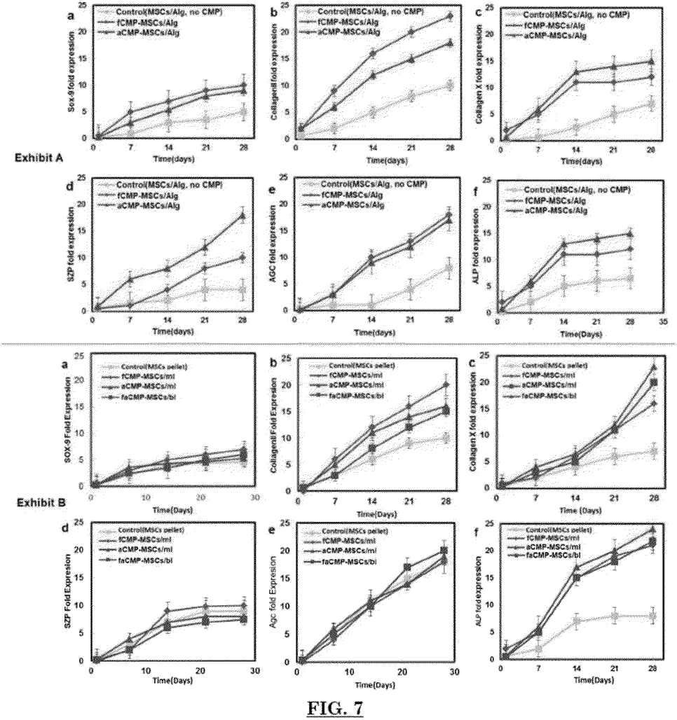

An injectable method was developed for the delivery of CMP-MSCs to the regeneration site. In the injectable method, fetal or adult CMPs were dispersed in an alginate precursor solution, the suspension was injected in a mold, and cross-linked with CaCl₂ as the chelating agent. FIG. 6 at a shows the effect of CMP size on gelation time of the alginate for different concentrations of CaCl₂. The pristine alginate solution had the fastest gelation time compared to the solutions with CMP-MSCs for all CaCl₂ concentrations. For a given CMP size, the gelation time decreased almost linearly with increasing CaCl₂ concentration. For a given CaCl₂ concentration, the gelation time increased with increasing CMP particle size. FIG. 6 at b shows the effect of CMP loading in the alginate solution on gelation time for different concentrations of CaCl₂. For a given CaCl₂ concentration, as CMP loading was increased from 30% to 70% by volume, the gelation time significantly decreased. In general, the gelation times were in the range of 1-4 min, which were within the clinically acceptable range for in-situ gelling injectable implants. See E. Ruvinov, T. Tavor Re’em, F. Witte, S. Cohen, Articular cartilage regeneration using acellular bioactive affinity-binding alginate hydrogel: A 6-month study in a mini-pig model of osteochondral defects, J Orthop Translat 16 (2019) 40-52. The graphs in exhibit A of FIG. 7 compare mRNA expression of chondrogenic markers for adult or fetal CMP-MSCs/alg hydrogels as a function of incubation time in chondrogenic medium. The control group in exhibit A was MSCs encapsulated directly, without CMPs, in alginate. The mRNA expressions included Sox-9, SZP as the superficial zone marker, see K. Miyatake, K. Iwasa, S.M. McNary, G. Peng, A.H. Reddi, Modulation of Superficial Zone Protein/Lubricin/PRG4 by Kartogenin and Transforming Growth Factor-beta1 in Surface Zone Chondrocytes in Bovine Articular Cartilage, Cartilage 7(4) (2016) 388-97. Col II and AGC as the middle zone markers, and Col X and ALP as markers of cartilage hypertrophy in the calcified zone. See, S. Diederichs, Y. Renz, S. Hagmann, B. Lotz, E. Seebach, W. Richter, Stimulation of a calcified cartilage connecting zone by GDF-5-augmented fibrin hydrogel in a novel layered ectopic in vivo model, J Biomed Mater Res B Appl Biomater 106(6) (2018) 2214-2224. For all markers and culture times, the marker expressions for aCMP-MSCs and fCMP-MSCs were significantly higher than the control group in which MSCs were directly encapsulated in alginate. Sox-9, AGC, Col X and ALP expressions of aCMP-MSCs/alg were not significantly different from fCMP-MSCs whereas SZP was higher and Col II was lower. We previously reported that zone-specific growth factors play a key role in chondrogenic differentiation of MSCs to zone-specific phenotypes, see T. Karimi, D. Barati, O. Karaman, S. Moeinzadeh, E. Jabbari, A developmentally inspired combined mechanical and biochemical signaling approach on zonal lineage commitment of mesenchymal stem cells in articular cartilage regeneration, Integrative Biology 7(1) (2014) 112-127. As the culture medium was not supplemented with zone-specific growth factors, we did not expect a significant difference in the expression of zone-specific markers between aCMP-MSCs and fCMP-MSCs. The data in exhibit A, see FIG. 7, demonstrate that the adult or fetal CMPs augmented chondrogenic differentiation of MSCs encapsulated in alginate hydrogel, as compared to the commonly used method of direct injection of MSCs in alginate hydrogel, for repair of articular cartilage defects. See L.X. Tay, R.E. Ahmad, H. Dashtdar, K.W. Tay, T. Masjuddin, S. Ab-Rahim, P.P. Chong, L. Selvaratnam, T. Kamarul, Treatment outcomes of alginate-embedded allogenic mesenchymal stem cells versus autologous chondrocytes for the repair of focal articular cartilage defects in a rabbit model, Am J Sports Med 40(1) (2012) 83-90.

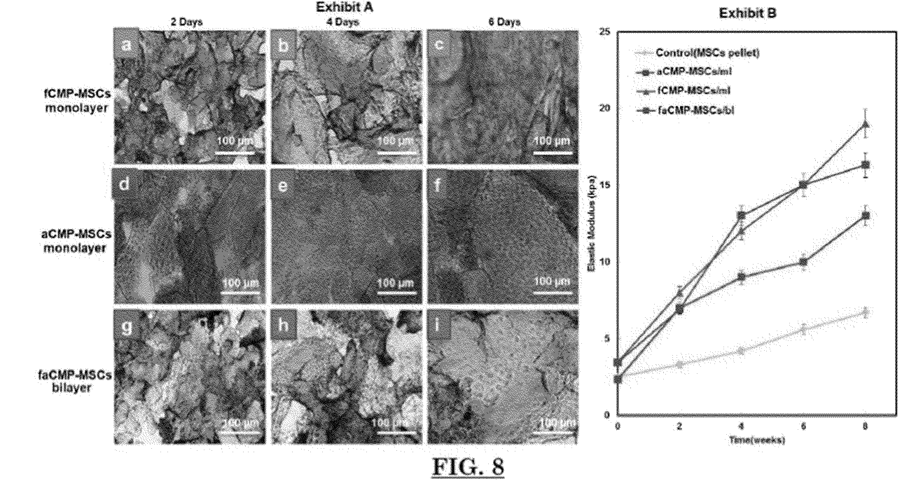

An implantable cell sheet method was also developed for the delivery of CMP-MSCs to the regeneration site. In this method, mono- or bilayer cell sheets were generated by sequential gravitational settling of aCMP-MSCs or fCMP-MSCs and incubation in MSC medium for 48 h. Next, the cell sheets were cultured in chondrogenic medium for up to 28 day to induce chondrogenesis and articular cartilage tissue formation. To demonstrate ECM secretion and fusion of CMP-MSCs with incubation in chondrogenic medium, at different time points, the cultured fCMP-MSCs, aCMP-MSCs, and faCMP-MSCs cell sheets were stained with blue, red and purple dyes, respectively, and imaged with a microscope. The blue, red and purple images in Exhibit A of FIG. 8 show the steady fusion and disappearance of the interface between CMP-MSCs particles with incubation time from 2 to 6 days for fetal and adult monolayers, and fetal/adult bilayer, respectively. FIG. 8 at Exhibit B compares the steady increase in elastic modulus of CMP-MSCs cell sheets with MSC pellets (without CMPs). The elastic modulus of all CMP-MSCs cell sheets and the MSC pellet steadily increased with incubation time. At any culture time, the elastic modulus of CMP-MSCs cells sheets was higher than the MSC pellet, which was attributed to their higher extent of chondrogenic differentiation of MSCs and ECM secretion, and fusion of CMPs. Further, the elastic modulus of fCMP-MSCs monolayer and faCMP-MSCs bilayer was higher than aCMP-MSCs monolayer after 8 weeks of incubation.

The graphs in Exhibit B of FIG. 7 compare mRNA expression of chondrogenic markers for aCMP-MSCs/ml, fCMP-MSCs/ml, and faCMP-MSCs/bl as a function of incubation time in chondrogenic medium. The control group in exhibit B was the commonly used 3D pellet culture formed by direct centrifugation of MSCs suspended in medium, as described, see H. Rogan, F. Ilagan, F. Yang, Comparing Single Cell Versus Pellet Encapsulation of Mesenchymal Stem Cells in Three-Dimensional Hydrogels for Cartilage Regeneration, Tissue Eng Part A 25(19-20) (2019) 1404-1412. For Sox-9, SZP and AGC markers, the expressions for CMP-MSCs monolayers and bilayers were close to the control group expressions; and for Col II, Col X and ALP makers, the expressions for CMP-MSCs were significantly higher than the control. There was not a significant difference in the expression of markers between the adult and fetal CMP-MSCs cell sheets, except for Col II marker expression of fCMP-MSCs/ml, which was slightly higher than the faCMP-MSCs/bl. The data in Exhibit B of FIG. 7 demonstrate that the adult or fetal CMP-MSCs cell sheet is as good, if not better, than the commonly used MSC pellets, see Id. The data in FIGS. 5-7 show that MSCs can be expanded on adult or fetal CMPs and the expanded CMP-MSCs can potentially be injected or implanted in an articular cartilage defect without the need to detach or separate MSCs from CMPs. Further, the experimental results indicate that the adult or fetal CMPs, as an articular cartilage mimetic substrate, augment chondrogenic differentiation and maturation of MSCs.

The compressive modulus of adult and fetal CMP-MSCs/alg hydrogels was measured as a function of percent CMPs (based on alginate weight) with incubation time in chondrogenic medium and the results are shown in FIG. 8; the moduli of aCMP-MSCs/alg with average CMP size of 90, 190 and 250 µm are shown in FIG. 8 at a-c, respectively; and the moduli of fCMP-MSCs are shown in FIG. 8 at d-f. The compressive moduli of all samples including the control group, in which MSCs were directly encapsulated in alginate, increased with incubation time. For all CMP groups, adult or fetal, the compressive modulus was highest at the time point of 8 weeks. For a given incubation time, CMP percent and CMP size, the compressive moduli of CMP-MSCs/alg groups were significantly higher than that of MSCs directly encapsulated in alginate (control group, yellow curve) for adult or fetal CMPs. For adult or fetal CMPs, the moduli of CMP-MSCs/alg groups with CMP percent of 50% and 70% were higher than the 30% group for all CMP sizes and all incubation times. For adult or fetal CMPs, the moduli of CMP-MSCs/alg groups with particle sizes of 190 µm and 250 µm were higher than the 90 µm size group for all CMP loadings and all incubation times. For adult CMPs, the CMP-MSCs/alg with CMP size of 250 µm with 50% CMP loading after 8 weeks incubation had the highest compressive modulus of 238±10 kPa followed by the group with CMP size of 190 µm with 50% CMP loading at 218±10 kPa. For fetal CMPs, the CMP-MSCs/alg groups with CMP sizes 250 µm and 190 µm with 50% CMP loading after 8 weeks incubation had the highest modulus at 197±20 kPa. Overall, the adult CMP-MSCs with 250 µm CMP size and 50% loading and 8 weeks of incubation had the highest compressive modulus. According to previous studies, the extent of chondrogenic differentiation of MSCs increases with cell-cell contact, see B. Cao, Z. Li, R. Peng, J. Ding, Effects of cell-cell contact and oxygen tension on chondrogenic differentiation of stem cells, Biomaterials 64 (2015) 21-32. Therefore, the higher modulus of adult or fetal CMP-MSCs/alg groups with CMP sizes of 250 and 190 µm can be attributed to the higher number of cells per CMP, thus resulting in greater extent of cell-cell contact and chondrogenesis.

The compressive modulus of mono- and bi-layer cell sheets was measured as a function of CMP size with incubation time in chondrogenic medium and the results are shown in FIG. 8 at g-i; the moduli of adult and fetal CMP-MSCs/ml cell sheets as a function of CMP size is shown in graphs g and h, respectively; the moduli of adult and fetal CMP-MSCs/ml and faCMP-MSCs/bl are compared in graph i. The control group in FIG. 8 at g-i was MSCs in pellet culture, see H. Rogan et al. The modulus of all CMP-MSCs cell sheets increased monotonically with incubation time. For all time points, the moduli of CMP-MSCs cell sheets were significantly higher than the control group (MSC pellet). After 8 weeks of incubation, the moduli of adult or fetal CMP-MSCs cell sheets with CMP sizes of 250 and 190 µm were significantly higher than that of the 90 µm CMP size, see FIG. 8 at g and h, which was attributed to the greater extent of cell-cell contact for larger CMPs, see B. Cao, Z. Li, R. Peng, J. Ding, Effects of cell-cell contact and oxygen tension on chondrogenic differentiation of stem cells, Biomaterials 64 (2015) 21-32. For all time points, the moduli of aCMP-MSCs/ml and fCMP-MSCs/ml, produced from CMP-MSCs with equal parts of 90, 190 and 250 µm CMP, and faCMP-MSCs/bl cultured in chondrogenic medium were higher than the control group (MSC pellet). However, after 8 weeks of incubation, there was no difference between the moduli of aCMP-MSCs/ml, fCMP-MSCs/ml, and faCMP-MSCs/bl. The compressive moduli of CMP-MSCs monolayer and bilayer cell sheets were in the range of 250±30 kPa which was significantly higher than that of the MSC pellet at 70±5 kPa. Overall, after 8 weeks of incubation, the adult or fetal CMP-MSCs/ml cell sheets had a higher compressive modulus (250±30 kPa) compared to the CMP-MSCs/alg (238±10 kPa).

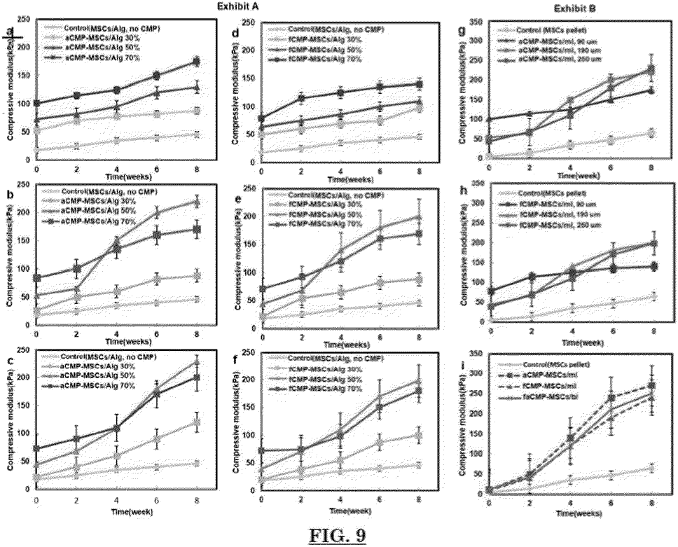

The viability of MSCs in CMP-MSCs/alg or CMP-MSCs cell sheets was imaged by cAM/EthD live/dead staining. The nonfluorescent Calcein AM is converted to green fluorescent calcein in live cells after hydrolysis by intracellular esterases. The weakly fluorescent EthD becomes intensely red fluorescent after binding to single-strand DNA of dead cells. Phalloidin/DAPI image the shape and position of viable cells within a matrix by staining the cytoskeleton’s actin fibers and the DNA in the cell nucleus, respectively. Exhibit A in FIG. 9 shows cAM/EthD staining for CMP-MSCs/alg and CMP-MSCs cell sheets, respectively; exhibit B of FIG. 9 shows the corresponding Phalloidin/DAPI staining. The first and second rows in exhibits A and B of FIG. 9 correspond to fetal and adult CMPs, respectively, whereas the third row is for fa/CMP-MSCs/bl cell sheet. The CMP loading in CMP-MSCs/alg hydrogels was 50% and the average size was 190 µm. The cell sheets in FIG. 9 were produced from CMP-MSCs with equal parts of 90, 190 and 250 µm CMPs. The CMP-MSCs/alg and cell sheets in FIG. 9 were cultured in chondrogenic medium for 21 days. The images in exhibit A show >99% cell viability for fetal or adult CMP-MSCs/alg, CMP-MSCs/ml and faCMP-MSCs/bl. The images in Exhibit B show extensive spreading and growth of spindle-shape MSCs in CMPs. No significant difference in cell morphology and shape was observed between adult or fetal CMP-MSCs/alg and CMP-MSCs cell sheets.

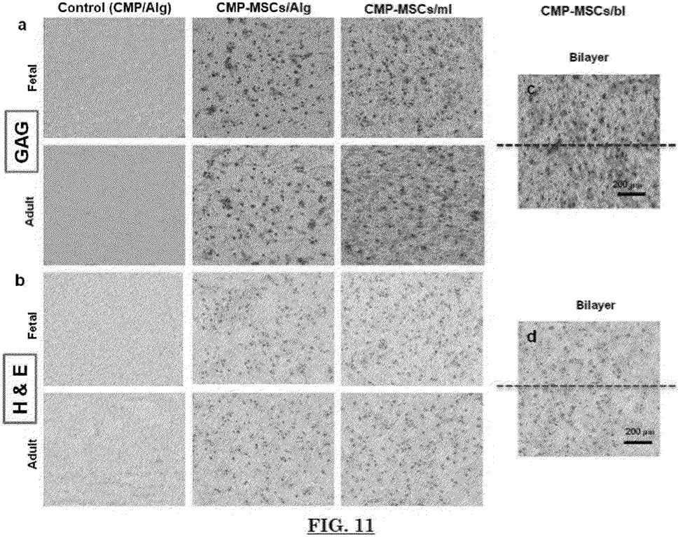

FIG. 11 shows cell morphology and GAG accumulation in histological sections of adult and fetal CMP-MSCs/alg, CMP-MSCs/ml, and faCMP-MSCs/bl groups stained with H&E and Alcian blue, respectively. The fetal or adult CMPs in alginate hydrogel without MSCs was used as the control group. The average size of CMPs for all groups was 190 µm. The percent CMP in alginate in CMP-MSCs/alg groups was 50%. There was no significant difference in cell morphology between CMP-MSCs/alg and CMP-MSCs cell sheets or between fetal and adult groups. The cell content and GAG intensity of CMP-MSCs cell sheets were slightly higher than CMP-MSCs/alg. Further, the CMP-MSCs cell sheets had a more uniform cell and GAG distribution as compared to CMP-MSCs/alg.

### Conclusion

This work describes a novel process to produce mono- or multi-layer cell sheets from fetal or adult articular matrix for regeneration of articular cartilage defects. First, fetal or adult bovine articular cartilage was minced, decellularized, and freeze-dried. Next, the freeze-dried extracellular matrix (ECM) was grinded and sieved to produce microparticles (CMPs) with average sizes in the range of 90-250 µm. Then, human MSCs were seeded on CMPs and expanded in a bioreactor to generate CMP-MSCs. MSCs expanded on fetal or adult CMPs in basal medium maintained their mesenchymal marker expression. Next, two approaches were used to generate cellular constructs for injection or implantation in a full-thickness articular cartilage defect. In one approach, CMP-MSCs were suspended in sodium alginate, injected into a mold, cross-linked with calcium chloride, and incubated in chondrogenic medium to generate an injectable construct for articular cartilage regeneration. In another approach, CMP-MSCs were suspended in a culture medium, allowed to settle on a mold surface by gravitational force, and fused by incubation in chondrogenic medium to generate an implantable cell sheet for articular cartilage regeneration.

Multilayer cell sheets can be generated by sequential settling and fusion of zone-specific CMP-MSCs to simulate the stratified structure of articular cartilage. Fetal or adult CMP-MSCs in alginate hydrogels showed significantly higher expression of chondrogenic markers after four weeks and compressive modulus after eight weeks of incubation in chondrogenic medium compared to MSCs directly encapsulated in alginate gels; CMP-MSCs cell sheets showed significantly higher expression of chondrogenic markers after four weeks and compressive modulus after eight weeks of incubation in chondrogenic medium compared to MSCs in a pellet culture. The higher quality of the generated tissues for CMP-MSCs groups was attributed to superior allocation of cell-cell and cell-matrix interactions compared to MSCs encapsulated in alginate or the MSC pellet. The cell sheet approach can be used to produce multilayer constructs with individual layers mimicking composition and cellularity of distinct zones of articular cartilage.

### Figure Legends

FIG. 1 shows - a schematic representation for production of injectable or implantable CMP-MSCs. (a) Fetal or adult articular cartilage is harvested from bovine cadaver, dissected and minced, decellularized, and freeze-dried. The freeze-dried fragments were grinded in liquid nitrogen and sieved to produce fetal or adult cartilage microparticles (CMPs) with average sizes of 90, 190 and 250 µm; (b) MSCs were seeded on the CMPs and expanded in a tissue culture bioreactor. In one approach, the CMP-MSCs were suspended in alginate gel to generate an injectable hydrogel for treatment of irregularly shaped articular cartilage defects. In another approach, the CMP-MSCs were suspended in medium, allowed to settle by the force of gravity on the surface of a mold, and incubated to generate an implantable cell sheet.

FIG. 2 shows the size distribution of adult (a-c) and fetal (d-f) CMPs. The distributions a, b and c correspond to adult CMPs with average size of 90, 190 and 250 µm, respectively; distributions d, e and f correspond to fetal CMPs with average size of 90, 190 and 250 µm. The insets in the distributions are SEM images of the CMPs showing the irregular geometry of the particles.

FIG. 3 shows growth of MSCs on the fetal (a) and adult (b) CMPs with incubation time in basal medium in a tissue culture bioreactor for CMP particle sizes of 90 µm (red), 190 µm (green) and 250 µm (blue). MSCs grown on 2D tissue culture plates (orange) and 3D polystyrene beads (purple) were used as controls; (c) comparison of MSC growth on fetal (blue) and adult (red) CMPs with incubation time in basal medium for CMP particle sizes of 90 µm (light), 190 µm (medium) and 250 µm (dark).

FIG. 4 shows the live (green) and dead (red) fluorescent images of a randomly selected microparticle from fetal and adult CMP-MSCs with incubation time (1, 7, 14, 21 days) in basal medium for CMP particle sizes of 90, 190 and 250 µm. The control group (most left column) is the image of a microparticle without incubation with MSCs. The images in last column on the right are 5x magnification of the 21 days images. The scale bar in the images is 100 µm.

FIG. 5 shows the mRNA expression of markers Sox-9 (a), Col I (b), CD34 (c), Col II (d), CD105 (e), CD166 (f), Cd44 (g), and CD45, AGC, Scx (h) of MSCs cultured on adult (red) and fetal (blue) CMPs as a function of incubation time in basal medium for up to 21 days. The control group (yellow) is MSCs cultured on 2D tissue culture plates.

FIG. 6 at (a) Gelation time of alginate gels as a function of CaCl₂ concentration containing 50% (by alginate weight) CMPs with particle size of 90 µm (red), 190 µm (green), and 250 µm (blue); (b) Gelation time of alginate gels as a function of CaCl₂ concentration containing with 30% (very light purple), 50% (light purple) and 70% (purple) CMPs with average size of 190 µm. The control group in (a,b) is alginate gel without CMPs.

FIG. 7 at (Exhibit A) shows the mRNA expression of chondrogenic markers Sox-9 (a), Col II (b), Col X (c), SZP (d), AGC (e), and ALP (f) with incubation time for fCMP-MSCs (blue) or aCMP-MSCs (red) encapsulated in injectable alginate hydrogels and incubated in chondrogenic medium for up to 28 days; the control group (yellow) in Exhibit A is MSCs (without CMP) encapsulated in alginate hydrogel and incubated in chondrogenic medium. (Exhibit B) The mRNA expression of chondrogenic markers Sox-9 (a), Col II (b), Col X (c), SZP (d), AGC (e), and ALP (f) with incubation time for CMP-MSCs implantable cell sheets and incubated in chondrogenic medium for up to 28 days; groups include monolayer fCMP-MSCs (blue), monolayer aCMP-MSCs (red), and bilayer faCMP-MSCs (purple) cell sheets; the control group (yellow) in Exhibit B is the MSC pellet cultured in chondrogenic medium.

FIG. 8 at Exhibit A shows images of monolayer fCMP-MSCs (blue), monolayer aCMP-MSCs (red), and bilayer faCMP-MSCs (purple) cell sheets after 2, 4, and 6 days incubation in chondrogenic medium stained with blue, red, and purple dyes, respectively, showing the fusion of CMP-MSCs with time; the scale bar in the images is 100 µm. Exhibit B shows elastic modulus of fCMP-MSCs (blue), aCMP-MSCs (red), and bilayer faCMP-MSCs (purple) cell sheets with incubation time in chondrogenic medium for up to 6 days showing the increase in modulus with CMP-MSCs fusion; the control group in Exhibit B is the MSC pellet cultured in chondrogenic medium.

FIG. 9 at Exhibit A shows compressive modulus of aCMP-MSCs/Alg (left column) and fCMP-MSCs/Alg (right column) with incubation time in chondrogenic medium for CMPs as a function of percent CMP and average CMP size; CMP percentage in Exhibit A were 30% (light shade), 50% (medium shade), and 70% (dark shade). FIG. 9 at a, b, and c in Exhibit A are for aCMP-MSCs/alg with average CMP sizes of 90 µm (a), 190 µm (b) and 250 µm (c), respectively whereas FIG. 9 at d, e, and f are for fCMP-MSCs/alg; the control group (yellow) in Exhibit A is MSCs (without CMP) encapsulated in alginate hydrogel and incubated in chondrogenic medium. (Exhibit B) Compressive modulus of aCMP-MSCs/ml (g) and fCMP-MSCs/ml (h) with incubation time in chondrogenic medium as a function of average CMP sizes of 90 µm (red), 190 µm (green) and 250 µm (blue); (i) Comparison of compressive moduli of aCMP-MSCs/ml (dash red), fCMP-MSCs/ml (dash blue), and faCMP-MSCs/bl (solid purple) cell sheets as a function of incubation time in chondrogenic medium; the control group (yellow) in Exhibit B is MSC pellet cultured in chondrogenic medium.

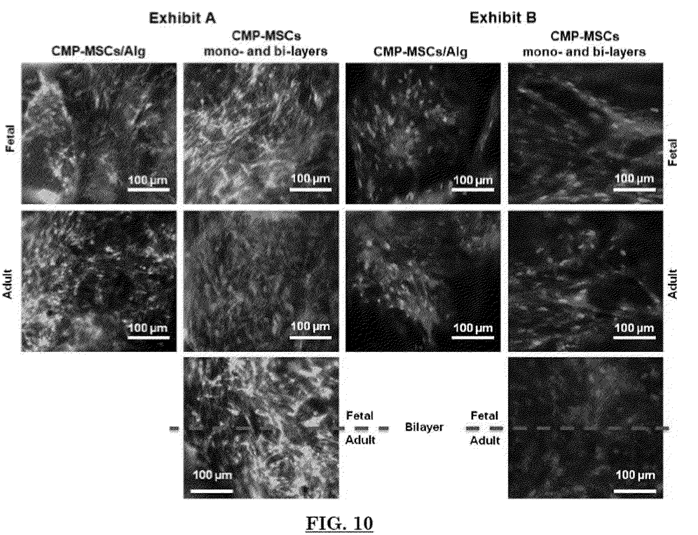

FIG. 10 at Exhibit A shows Calcein AM (green) and EthD (red) fluorescent images of live and dead MSCs, respectively, in CMP-MSCs/alg hydrogels (left column) and CMP-MSCs cell sheets (right column) after 21 days incubation in chondrogenic medium. (Exhibit B) Phalloidin and DAPI stained images showing cytoskeletal and nuclear compartments of MSCs, respectively, in CMP-MSCs/alg hydrogels (left column) and CMP-MSCs cell sheets (right column) after 21 days incubation in chondrogenic medium. The first and second columns in the exhibits are for fCMP-MSCs and aCMP-MSCs, respectively, and the third row is for bilayer faCMP-MSCs cell sheets. The scale bar in the images of Exhibits A and B are 100 µm.

FIG. 11 shows Alcian blue (a) and H&E (b) stained histological sections of CMP-MSCs/alg hydrogel (second column), CMP-MSCs/ml (third column) and CMP-MSCs/bl cell sheets after four weeks of incubation in chondrogenic medium; the first and third rows are for fetal CMPs whereas the second and fourth rows are for adult CMPs; the control group for CMP-MSCs hydrogel was MSCs (without CMP) directly encapsulated in alginate and cultured in chondrogenic medium whereas the control group for CMP-MSCs/ml and CMP-MSCs/bl was MSC pellet cultured in chondrogenic medium. The scale bar in the images is 200 µm.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for forming a novel monolayer implant construct comprising:
forming at least one nanogel in at least one microcapsule via;
chain extending at least one first polyethylene oxide macromer with at least one lactide-glycolide;
terminating at least one chain end with an acrylate functional group;
crosslinking the at least one first polyethylene oxide macromer with at least one lactide-glycolide terminated on at least one chain end with an acrylate functional group with a second polyethylene oxide macromer with at least one lactide-glycolide terminated on at least one chain end with an acrylate functional group to form at least one nanogel; and
conjugating at least one morphogen to the at least one nanogel to form at least one morphogen-encapsulated nanogel;
forming at least one cartilage microparticle from articular cartilage;
transferring the at least one cartilage microparticle to a cell culture bioreactor containing at least one cell culture medium wherein at least one cell adheres to the at least one cartilage microparticle;
forming a suspension comprising the at least one cartilage microparticle with at least one cell adhered, the at least one morphogen-encapsulated nanogel, and at least one crosslinking agent in a tissue culture medium; and
employing a cross-linking initiator to form a cross-linked monolayer implant.

2. The method of claim 1, wherein release of the at least one morphogen is controlled via changing a composition of the nanogel to change a release duration of the at least one morphogen.

3. The method of claim 1, wherein the articular cartilage is harvested from frozen human cadaver or animal tissue.

4. The method of claim 1, wherein the at least one cartilage microparticle is decellularized.

5. The method of claim 1, wherein the at least one cartilage microparticle ranges in size from 50 to 500 µm.

6. The method of claim 1, wherein the at least one cell culture medium comprises at least one mesenchymal stem cell.

7. The method of claim 1, wherein the suspension is injected at a tissue injury site prior to employing the cross linking initiator.

8. A method for forming an implantable microgel-cell construct for a zonally structured tissue comprising:
forming at least one nanogel in at least one microcapsule via;
chain extending at least one first polyethylene oxide macromer with at least one lactide-glycolide;
terminating at least one chain end with an acrylate functional group;
crosslinking the at least one first polyethylene oxide macromer with at least one lactide-glycolide terminated on at least one chain end with an acrylate functional group with a second polyethylene oxide macromer with at least one lactide-glycolide terminated on at least one chain end with an acrylate functional group to form at least one nanogel; and
conjugating at least one morphogen to the nanogel to form at least one morphogen-encapsulated nanogel;
forming at least one microparticle;
transferring the at least one microparticle to a cell culture bioreactor containing at least one cell culture medium wherein at least one cell adheres to the at least one microparticle;
forming a first suspension comprising the at least one microparticle with at least one cell adhered and the at least one morphogen-encapsulated nanogel;
allowing the first suspension to settle gravitationally on a surface and fuse via secretion of an extracellular matrix tor form a first monolayer;
forming a second suspension comprising the at least one microparticle with at least one cell adhered and the at least one morphogen-encapsulated nanogel; and
allowing the second suspension to settle gravitationally on a surface and fuse via secretion of an extracellular matrix or form a second monolayer atop the first monolayer to form a multilayer construct.

9. The method of claim 8, wherein the first monolayer ranges from 100 to 500 µm in thickness.

10. The method of claim 8, wherein the first monolayer and the second monolayer are formed with different extracelluar matrix compositions and/or different morphogen nanogels.

11. The method of claim 8, wherein the multilayer construct comprises three or more monolayers.

12. The method of claim 11 , wherein the at least three or more monolayers are formed with different extracelluar matrix compositions and/or different morphogen nanogels.

13. The method of claim 12, wherein the suspension is transferred to a mold with a predefined shape.

14. The method of claim 8, wherein release of the at least one morphogen is controlled via changing a composition of the nanogel to change a release duration of the at least one morphogen.

15. The method of claim 8, wherein the at least one microparticle is decellularized.

16. The method of claim 8, wherein the at least one microparticle ranges in size from 50 to 500 µm.

17. The method of claim 8, wherein the at least one cell culture medium comprises at least one mesenchymal stem cell.

18. A novel multilayer cellular construct comprising:
at least one multilayer cellular construct comprising at least one decellularized cell-seeded fused microcarrier, wherein each layer of the at least one multilayer cellular construct mimics biochemical and cellular properties of at least one zone of a zonally structured tissue.

19. The novel multilayer cellular construct of claim 1, wherein the decellularized cell-seeded fused microcarrier comprises at least one cartilage microp article.

20. The novel multilayer cellular construct of claim 1, wherein the at least one cartilage microparticle comprises either human or animal articular cartilage.

21. The novel multilayer cellular construct of claim 1, wherein the at least one cartilage microcarrier ranges in size from 50 to 500 µm.

22. The novel multilayer cellular construct of claim 1, wherein the at least one multilayer cellular construct is used to regenerate heart, skin, articular cartilage, blood vessel, nerve conduit, ligament and/or tendon tissue.


SEQUENCE LISTING (21 sequences)

SEQ ID NO: 1 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHHVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYNNATNHAIVQTLVHFINPETVPKPCCAPTQLNAISVLYFDDSSNVILKKYRNMVVRACGCH

SEQ ID NO: 3 - protein, 398 aa, Homo sapiens
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPVMSNGSLSPPIPRTSNKVPVVQPSHAVHPLTPLITYSDEHFSPGSHPSHIPSDVNSKQGMSRHPPAPDIPTFYPLSPGGVGQITPPLGWQGQPVYPITGGFRQPYPSSLSVDTSMSRFSHHMIPGPPGPHTTGIPHPAIVTPQVKQEHPHTDSDLMHVKPQHEQRKEQEPKRPHIKKPLNAFMLYMKEMRANWAECTLKESAAINQILGRRWHALSREEQAKYYELARKERQLHMQLYPGWSARDNYGKKKKRKREKLQESASGTGPRMTAAYI

SEQ ID NO: 4 - protein, 70 aa, Homo sapiens
GPETCLGAELVDALQFVCGDRGFYFNKPTGYGSSSRRAPQTGIVDECCFRSCDLRRLEMYCAPLKPAKSA

SEQ ID NO: 5 - protein, 86 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTP

SEQ ID NO: 6 - protein, 176 aa, Homo sapiens
IIGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGG

SEQ ID NO: 7 - protein, 311 aa, Homo sapiens
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQ

SEQ ID NO: 8 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 9 - protein, 264 aa, Homo sapiens
MKVFISADLEGVAGSTTWIETEKNTPDYAPFQQQMTKEVEAAIEGAIAGGATEILLKDAHDSARNIDISNLPENVKIIRGWTGDPMCMVAGLDASFDRAIFIGYHSKGGSHRNPLAHTLVVNADVKINGEYASEFLINTYAAALHGVPVAFVSGDVGLTEEIMTVNENIVTYATKEGIGGATLSVSPKLAISETKRLVEEAMKVEKNSLQVNLPEKFVVEIVYRDHTRAFRNSFYPGAKFKPHNTVEYETENYFDVLRILQFLT

SEQ ID NO: 10 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 11 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 12 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 13 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 14 - protein, 509 aa, Homo sapiens
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRP

SEQ ID NO: 15 - protein, 112 aa, Homo sapiens
MEKILILLLVALSVAYAAPGPRGIIINLENGELCMNSAQCKSNCCQHSSALGLARCTSMASENSECSVKTLYGIYYKCPCERGLTCEGDKTIVGSITNTNFGICHDAGRSKQ

SEQ ID NO: 16 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 17 - protein, 467 aa, Homo sapiens
MMAILDYWDGVLYYPILIIILTVAGLYFTGKTGFVQLRMFGESIRVVREKPATKGAVSSFQALMVSTASRVGTGNIIGVSTAICLGGPGAVFWMWLLALIGGSTAFIESTLAQIFKRRDVRGGSFGGPAYYIEAALHQRWLGVVFAIALILTYAGGFNLLCSYNMQSTFMAYSFYQPDSTPWIIGAIFALIVGYCLVGGGKRIIRITSVLVPVMGTVYIIAALLVILLNITALPHVFSVIFADAFDFQSILGGVSGSCMIYGIKRGLYSNEAGIGSAPNAAAAAMVSHPVKQGLVQMLSVFIDTLLVCSATAFLGLFSGVPATEEVAGALYIQQAATSVYGHFGPLLITICMLLFGFSTLIGNLYYVDNCIHFIHRKMPSQWFVILFRLVCIFIIFIGSAMSMAAVWDIADILMALMCIINIPACLLLGNMAVRALKDYQQQKKAGHDPVFKAADIGLSDDRVEFWK

SEQ ID NO: 18 - protein, 201 aa, Homo sapiens
MSFATLRPAPPGRYLYPEVSPLSEDEDRGSDSSGSDEKPCRVHAARCGLQGARRRAGGRRAGGGGPGGRPGREPRQRHTANARERDRTNSVNTAFTALRTLIPTEPADRKLSKIETLRLASSYISHLGNVLLAGEACGDGQPCHSGPAFFHAARAGSPPPPPPPPPARDGENTQPKQICTFCLSNQRKLSKDRDRKTAIRS

SEQ ID NO: 19 - protein, 680 aa, Homo sapiens
MLPQIPFLLLVSLNLVHGVFYAERYQMPTGIKGPLPNTKTQFFIPYTIKSKGIAVRGEQGTPGPPGPAGPRGHPGPSGPPGKPGYGSPGLQGEPGLPGPPGPSAVGKPGVPGLPGKPGERGPYGPKGDVGPAGLPGPRGPPGPPGIPGPAGISVPGKPGQQGPTGAPGPRGFPGEKGAPGVPGMNGQKGEMGYGAPGRPGERGLPGPQGPTGPSGPPGVGKRGENGVPGQPGIKGDRGFPGEMGPIGPPGPQGPPGERGPEGIGKPGAAGAPGQPGIPGTKGLPGAPGIAGPPGPPGFGKPGLPGLKGERGPAGLPGGPGAKGEQGPAGLPGKPGLTGPPGNMGPQGPKGIPGSHGLPGPKGETGPAGPAGYPGAKGERGSPGSDGKPGYPGKPGLDGPKGNPGLPGPKGDPGVGGPPGLPGPVGPAGAKGMPGHNGEAGPRGAPGIPGTRGPIGPPGIPGFPGSKGDPGSPGPPGPAGIATKGLNGPTGPPGPPGPRGHSGEPGLPGPPGPPGPPGQAVMPEGFIKAGQRPSLSGTPLVSANQGVTGMPVSAFTVILSKAYPAIGTPIPFDKILYNRQQHYDPRTGIFTCQIPGIYYFSYHVHVKGTHVWVGLYKNGTPVMYTYDEYTKGYLDQASGSAIIDLTENDQVWLQLPNAESNGLYSSEYVHSSFSGFLVAPM

SEQ ID NO: 20 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 21 - protein, 364 aa, Homo sapiens
MPQTVILPGPAPWGFRLSGGIDFNQPLVITRITPGSKAAAANLCPGDVILAIDGFGTESMTHADAQDRIKAAAHQLCLKIDRGETHLWSPQVSEDGKAHPFKINLESEPQDGNYFEHKHNIRPKPFVIPGRSSGCSTPSGIDCGSGRSTPSSVSTVSTICPGDLKVAAKLAPNIPLEMELPGVKIVHAQFNTPMQLYSDDNIMETLQGQVSTALGETPLMSEPTASVPPESDVYRMLHDNRNEPTQPRQSGSFRVLQGMVDDGSDDRPAGTRSVRAPVTKVHGGSGGAQRMPLCDKCGSGIVGAVVKARDKYRHPECFVCADCNLNLKQKGYFFIEGELYCETHARARTKPPEGYDTVTLYPKA